(19) United States
(12) Patent Application Publication
(10) Pub. No.: US 2019/0330641 A1
(43) Pub. Date: Oct. 31, 2019

(54) SYSTEMS FOR RECOMBINANT PROTEIN PRODUCTION

(71) Applicant: NATIONAL HELLENIC RESEARCH FOUNDATION, Athens (GR)

(72) Inventors: Georgios SKRETAS, Athens (GR); Dimitra GIALAMA, Athens (GR)

(73) Assignee: NATIONAL HELLENIC RESEARCH FOUNDATION, Athens (GR)

(21) Appl. No.: 16/310,595

(22) PCT Filed: Jun. 16, 2017
(86) PCT No.: PCT/EP2017/025168

§ 371 Date: Dec. 17, 2018

(30) Foreign Application Priority Data
Jun. 17, 2016 (EP) PCT/EP2016/001019

(51) Int. Cl. C12N 15/67; C07K 14/245

(52) U.S. Cl. CPC C12N 15/67; C07K 14/245

(57) ABSTRACT

The present invention relates to a method of producing a recombinant polypeptide in a genetically modified host cell. A host cell and a vector for transforming such host cell is also provided.

### FIELD OF THE INVENTION

The invention relates to the generation of certain strains, which can be generally utilized for achieving high-level production of recombinant proteins that are toxic and/or difficult to express in those strains or other prokaryotic or eukaryotic hosts.

### BACKGROUND TO THE INVENTION

Membranes constitute the border of cellular organelles and of the entire cell. Their purpose is to compartmentalize certain cell functions and to isolate them from their environment, but also to ensure regulated interaction with the surrounding space. Membrane proteins (MPs) are a major structural and functional component of biological membranes that mediate structural integrity, signaling, transport, energy production and more. The great importance of MPs is reflected by the fact that in both pro- and eukaryotes 20-30% of all genes encode such proteins. Furthermore, the proper folding and function of a wide variety of MPs are involved in devastating human diseases such as cystic fibrosis, Alzheimer's, lipodystrophy, cancer hypogonadotropic hypogonadism and more. Remarkably, MPs constitute about half of all current drug targets.

The development of novel therapeutic molecules that target MPs relies on the detailed understanding of MP structure and function. This, in turn, requires access to significant amounts of isolated protein, which will be utilized for biochemical studies, the development of MP-specific antibodies, and MP structure determination via X-ray crystallography or other methods. As MP natural abundance is usually very low, MPs are typically produced and isolated after recombinant overexpression in heterologous hosts, such as bacteria, yeasts, insect cells, mammalian cells or even transgenic animal. Each type of expression system has advantages and limitations, while there is really no way of predicting which MP will accumulate in higher amounts in which host.

The bacterium Escherichia coli (E. coli) has historically been the most popular and successful recombinant expression host for MP biochemical/structural studies. Solved structures of bacterially produced MPs correspond to sequences of both prokaryotic and eukaryotic origin, and include difficult targets, such as the mammalian G protein-coupled receptors (GPCRs) CXCR1 chemokine receptor and the neurotensin receptor 1. Despite these success stories, however, recombinant MP production in bacteria remains notoriously difficult and its success rather scarce. The problems associated with bacterial recombinant MP production are mainly three: (i) there is usually very little membrane-incorporated protein per cell, (ii) in cases where accumulation at the cell membrane occurs at appreciable levels, there is typically a very small amount of protein that is produced in a well folded and functional form, and (iii) overexpression is very frequently associated with severe cell toxicity, which further limits the volumetric protein yields.

Improvements of the expression host via genetic engineering or of the target MP itself via directed evolution have provided some solutions to the problem of low cellular productivity. The cytotoxicity caused by MP overexpression, however, is an issue which has not been systematically addressed. This is an important problem as MP-induced toxicity is observed routinely and is severe, oftentimes leading to complete growth arrest, very low levels of final biomass and significant reduction of total volumetric protein yields. A number of possible explanations have been suggested, such as collapse of the protein biosynthetic machinery as a result of high-level recombinant gene expression with concomitant degradation of ribosomal RNA and ribosome destruction; overloading and jamming of the SecYEG translocon resulting in compromised cell envelope and cytoplasmic proteomes and inefficient energy metabolism; or the actual biochemical and biophysical properties of the particular overexpressed MP. These hypotheses, however, have not yet been exploited to address the issue and improve recombinant MP production sufficiently.

Walker and coworkers utilized a difficult-to-express MP—the mitochondrial oxoglutarate malate carrier protein (OGCP)- to isolate E. coli BL21(DE3) mutant strains carrying spontaneously acquired suppressor mutations that alleviate the toxicity caused by OGCP production under the control of the strong T7 promoter. Two of the evolved strains, named C41 and C43, were found to be resistant to the toxicity caused by the production of a variety of membrane/soluble proteins and to allow increased biomass production, and are widely used for the production of hard-to-express and toxic proteins (mostly MPs). Many years later, de Gier and co-workers found that the mutations responsible for the suppressed toxicity were located in the sequence of lacUV5 promoter and that their effect was reduction of the expression levels and, thus, the overall translational efficiency of the T7 RNA polymerase, which in turn produced less mRNA corresponding to the target recombinant protein. These results revealed that there appears to be an optimal level of mRNA of the target gene that leads to maximal recombinant MP production, and further increases in target gene expression beyond that point disrupt cellular physiology and become deleterious for recombinant MP production as they decrease final biomass yields. In an improvement of the original strains reported many years later, De Gier and co-workers developed a system termed Lemo21(DE3), where the transcriptional activity of the T7 RNA polymerase is controlled by the cellular abundance of its inhibitor, T7 lysozyme, whose expression is in turn placed under the tight control of the rhamnose promoter. By varying the concentration of rhamnose, optimal induction conditions can be determined such that membrane protein yields can be maximized. The utility of the C41, C43 and Lemo21 strains is strictly limited to the use of the T7 promoter/T7 RNA polymerase system for expression of the target gene.

MPs constitute a large protein family involved in a number of functions with members that are significant drug targets for different diseases. Their overexpression is a major bottleneck in the pipeline of the generation of MP structures and rational drug design. In Escherichia coli, the main overexpression vehicle, the total yield of MP overexpression is typically low due to poor yields per cell and toxicity leading to low final biomass. The mechanism causing the toxicity of MP production remains unknown.

It will be understood by the skilled person that the invention is likely to also have utility in the expression of proteins other than MPs. Other classes of proteins which are targets for recombinant expression in bacterial and other hosts are soluble cytosolic proteins, soluble periplasmic proteins (proteins localized in the periplasm, i.e. the cellular compartment between the inner and outer membrane of Gram-negative bacteria), and secreted proteins (proteins destined for export to the extracellular space). Preferred proteins are those that are in some way toxic to the host, in particular E. coli, when they are overexpressed, i.e. they cause partial or complete growth arrest for the expression host upon induction of the overexpression process. Such proteins can be described as toxic cytoplasmic, periplasmic, or secreted soluble proteins. Alternatively, the target recombinant proteins may not be toxic, but their recombinant production is somehow problematic, i.e. satisfactory production yields cannot be achieved for these proteins in bacterial or other available cellular hosts. Examples of MPs are described in Table 1. Some examples of soluble cytosolic proteins are green fluorescent protein (GFP), thioredoxin 1 (TrxA), RraA, RraB, DnaK, DnaJ, GroEL, GroES, ClpB, trigger factor, Mn superoxide dismutase (SodA), Fe superoxide dismutase (SodB), β-galactosidase (LacZ) and NusA. Some examples of bacterial soluble periplasmic proteins are DsbA, DsbC, β-lactamase, Skp, and the maltose-binding protein (MalE). Some examples of secreted polypeptides are human growth hormone, follicle stimulating hormone (FSH), luteinizing hormone (LH), ghrelin, orexin, oxytocin, somatostatin, and thyroid-stimulating hormone.

TABLE 1
Examples of studied membrane proteins

| | | | Number | | |
|---|---|---|---|---|---|
| Membrane | | | of TM | | MW |
| protein | Organism | Function | helices | Topology | (kDa) |
| BR2 | Homo | Bradykinin receptor 2 | 7 | N^{out}-Cⁱⁿ | 44.5 |
| | sapiens | (GPCR) | | | |
| CB1 | H. sapiens | Central cannabinoid receptor | 7 | N^{out}-Cⁱⁿ | 52.9 |
| | | (GPCR) | | | |
| CB2 | H. sapiens | Peripheral cannabinoid | 7 | N^{out}-Cⁱⁿ | 39.7 |
| | | receptor (GPCR) | | | |
| NKR1 | H. sapiens | Neurokinin (substance P) | 7 | N^{out}-Cⁱⁿ | 46.3 |
| | | receptor 1 (GPCR) | | | |
| NTR1(D03) | Mus | Neurotensin receptor 1 - | 7 | N^{out}-Cⁱⁿ | 44.6 |
| | musculus | variant D03 (GPCR) | | | |
| SCD | H. sapiens | Stearoyl-CoA desaturase | 4 | Nⁱⁿ-Cⁱⁿ | 41.1 |
| MotA | E. coli | Stator element of the flagellar | 4 | Nⁱⁿ-Cⁱⁿ | 32.0 |
| | | motor complex. | | | |
| SapC | E. coli | Component of putative ABC | 5 | Nⁱⁿ-Cⁱⁿ | 31.5 |
| | | transporter SapABCDF | | | |
| YhaU | E. coli | Galactarate/glucarate/glycerate | 11 | Nⁱⁿ-Cⁱⁿ | 49.0 |
| (GarP) | | transporter | | | |
| YecS | E. coli | integral membrane subunit of | 3 | N^{out}-Cⁱⁿ | 24.8 |
| | | an L-cystine/L-cysteine ABC | | | |
| | | transporter complex | | | |
| CstA | E. coli | Peptide transporter | 18 | Nⁱⁿ-Cⁱⁿ | 75.1 |
| YidC | E. coli | Membrane protein integrase | 6 | Nⁱⁿ-Cⁱⁿ | 61.5 |

### SUMMARY OF THE INVENTION

The invention relates to the generation of E. coli strains, which can be generally utilized for achieving high-level production of recombinant MPs, or other recombinant proteins that are toxic and/or difficult to express in E. coli, or other prokaryotic or eukaryotic hosts. Our initial goal was to attempt to rewire the E. coli machinery to be able to withstand MP-induced toxicity and achieve high-level recombinant MP production. As the molecular sources of this phenomenon are not well understood, we employed a reverse-engineering approach and looked for single bacterial genes, whose co-expression can suppress MP-induced toxicity. After carrying out a genome-wide screen, we identified two highly potent effector genes: rraA, the gene encoding for RraA, an inhibitor of the mRNA-degrading activity of the E. coli RNase E and djlA, the gene encoding for the membrane-bound DnaK co-chaperone DjlA. E. coli strains co-expressing djlA and rraA were found to accumulate significantly higher levels of final biomass and to produce dramatically enhanced yields for a variety of recombinant proteins of both prokaryotic and eukaryotic origin compared to the parental strains.

Suppressor genes of the toxicity caused by MP overexpression were screened using the human G protein-coupled receptor (GPCR) bradykinin receptor 2 (BR2) as our model MP and it is shown for the first time that a membrane-bound co-chaperone, DjlA, or an inhibitor of RNase E, RraA, can enhance MP production in E. coli. They both suppress toxicity caused by BR2 overexpression and increase BR2 expression levels per cell dramatically. Furthermore, the suppressors enhance the production of a number of GPCRs and other toxic or non-toxic eukaryotic and bacterial MPs. In addition, insights into the mechanism of action of DjlA were gained as it was shown that its interaction with the Hsp70 chaperone DnaK is absolutely required for the observed improved phenotype. Apart from the general application of our effectors to MPs, we expect our screen to be applied to other cases of soluble E. coli toxic or non-toxic proteins facilitating the study of these ‘difficult’ targets.

We have discovered two genes of E. coli as suppressors of MP-induced toxicity:

(i) rraA, the gene encoding for RraA, an inhibitor of the mRNA-degrading activity of the E. coli RNase E, and
(ii) djlA, the gene encoding for the membrane-bound DnaK co-chaperone DjlA.

Co-expression of djlA and rraA in E. coli cells overexpressing BR2 (a membrane protein) on LB agar plates resulted in the formation of colonies with dramatically increased size compared to cells co-expressing a randomly selected gene contained in the ASKA library (FIG. 1B).

Co-expression of either gene dramatically improves the growth of the cells overexpressing BR2. In addition, the expression levels of BR2 on a per-cell basis are greatly enhanced in the presence of overexpressed djlA and rraA. The impact of these effectors was general as they improve the overexpression of a number of GPCRs and other toxic and non-toxic eukaryotic and prokaryoitc MPs.

When overexpression of the target protein was carried out with co-expression of djlA, or rraA, or both djlA with rraA, this resulted in about 30 and 50% increase in final culture density. Importantly, the suppressing effects of djlA and rraA on BR2 overexpression toxicity were found to be independent of the choice of the promoter and the plasmid, which are utilized for BR2, djlA and rraA production: when BR2-GFP expression was placed under the control of the tet promoter in the high-copy number vector pASK75 (pASKBR2-GFP vector; Table 3) and the expression of djlA and rraA was placed under the control of the araBAD promoter in the low-copy number plasmid pBAD33, we observed a large increase in final cell density of approximately 150% and 50% in the presence of djlA and rraA co-expression, respectively (FIG. 1C, left). The observed growth phenotypes did not occur due to a general growth-promoting effect of djlA and rraA overexpression. On the contrary, djlA overexpression has been found to be toxic for E. coli, an effect which was also observed here (FIG. 1C, right). Furthermore, when BR2 was produced in a form where GFP had been replaced with a hexa-histidine tag, djlA and rraA co-expression resulted in similar enhancements of final cell densities, thus demonstrating that the observed suppressing effects occur independently of the presence of the C-terminal fusion partner.

The nucleotide sequence of the E. coli djlA gene is:

atgCAGTATT GGGGAAAAAT CATTGGCGTG GCCGTGGCCT
TACTGATGGG CGGCGGCTTT TGGGGCGTAG TGTTAGGCCT
GTTAATTGGC CATATGTTTG ATAAAGCCCG TAGCCGTAAA
ATGGCGTGGT TCGCCAACCA GCGTGAGCGT CAGGCGCTGT
TTTTTGCCAC CACTTTTGAA GTGATGGGGC ATTTAACCAA
ATCCAAAGGT CGCGTCACGG AGGCTGATAT TCATATCGCC
AGCCAGTTGA TGGACCGAAT GAATCTTCAT GGCGCTTCCC
GTACTGCGGC GCAAAATGCG TTCCGGGTGG GAAAATCAGA
CAATTACCCG CTGCGCGAAA AGATGCGCCA GTTTCGCAGT
GTCTGCTTTG GTCGTTTTGA CTTAATTCGT ATGTTTCTGG
AGATCCAGAT TCAGGCGGCG TTTGCTGATG GTTCACTGCA
CCCGAATGAA CGGGCGGTGC TGTATGTCAT TGCAGAAGAA
TTAGGGATCT CCCGCGCTCA GTTTGACCAG TTTTTGCGCA
TGATGCAGGG CGGTGCACAG TTTGGCGGCG GTTATCAGCA
GCAAACTGGC GGTGGTAACT GGCAGCAAGC GCAGCGTGGC
CCAACGCTGG AAGATGCCTG TAATGTGCTG GGCGTGAAGC
CGACGGATGA TGCGACCACC ATCAAACGTG CCTACCGTAA
GCTGATGAGT GAACACCATC CCGATAAGCT GGTGGCGAAA
GGTTTGCCGC CTGAGATGAT GGAGATGGCG AAGCAGAAAG
CGCAGGAAAT TCAGCAGGCA TATGAGCTGA TAAAGCAGCA
GAAAGGGTTT AAAtga

The amino acid sequence of the E. coli DjlA protein is:

MQYWGKIIGV AVALLMGGGF WGVVLGLLIG HMFDKARSRK
MAWFANQRER QALFFATTFE VMGHLTKSKG RVTEADIHIA
SQLMDRMNLH GASRTAAQNA FRVGKSDNYP LREKMRQFRS
VCFGRFDLIR MFLEIQIQAA FADGSLHPNE RAVLYVIAEE
LGISRAQFDQ FLRMMQGGAQ FGGGYQQQTG GGNWQQAQRG
PTLEDACNVL GVKPTDDATT IKRAYRKLMS EHHPDKLVAK
GLPPEMMEMA KQKAQEIQQA YELIKQQKGF K

The nucleotide sequence of the E. coli rraA gene is:

atgAAATACG ATACTTCCGA GCTTTGTGAC ATCTATCAAG
AAGATGTTAA CGTCGTGGAA CCGCTGTTCT CCAACTTTGG
CGGACGGGCG TCGTTTGGCG GACAAATAAT CACGGTAAAA
TGTTTCGAGG ACAACGGGTT GCTGTACGAT CTGCTCGAAC
AGAATGGCCG TGGTCGTGTT CTTGTCGTCG ATGGCGGTGG
TTCTGTTCGT CGCGCACTGG TCGATGCTGA ACTGGCGCGT
CTGGCAGTAC AAAATGAATG GGAAGGTCTG GTCATTTACG
GCGCGGTGCG TCAGGTAGAT GACCTGGAAG AGTTGGATAT
CGGCATCCAG GCGATGGCGG CAATTCCGGT TGGTGCCGCT
GGCGAAGGCA TTGGCGAAAG CGATGTCCGC GTCAATTTTG
GCGGTGTCAC CTTCTTCTCC GGCGACCATC TTTATGCCGA
CAATACCGGG ATTATTCTTT CAGAAGATCC GCTGGATATT
GAAtg a

The amino acid sequences of the E. coli RraA is:

MKYDTSELCD IYQEDVNVVE PLFSNFGGRA SFGGQIITVK
CFEDNGLLYD LLEQNGRGRV LVVDGGGSVR RALVDAELAR
LAVQNEWEGL VIYGAVRQVD DLEELDIGIQ AMAAIPVGAA
GEGIGESDVR VNFGGVTFFS GDHLYADNTG IILSEDPLDI
E

The sequences of DjlA and RraA can also be found at www.ecocyc.org, www.uniprot.org or other similar databases.

Therefore we present as a feature of the invention a host cell, wherein the host is genetically modified so as to express elevated levels of DjlA and/or RraA, or variants thereof, relative to the expression of said protein in a wild-type strain.

As an alternative feature of the invention we present a method of producing a recombinant polypeptide in a host cell, wherein the host is genetically modified so as to express elevated levels of DjlA and/or RraA, or variants thereof, relative to the expression of said protein in a wild-type strain.

As an alternative feature of the invention we present a method of producing a recombinant polypeptide in a host cell comprising the steps of: (a) providing a nucleic acid comprising a sequence for the recombinant polypeptide and a nucleic acid sequence for either djlA and/or rraA, or variants thereof, operably linked to a promoter into an expression system; and (b) expressing the nucleic acid sequences of step (a) under conditions that allow expression of the polypeptide, thereby producing a recombinant polypeptide and either DjlA and/or RraA and, optionally, purifying the recombinant polypeptide.

As an alternative feature of the invention we present a method of transforming a host cell with (a) a nucleic acid comprising a sequence for a recombinant polypeptide and a sequence for either djlA, or a variant thereof, and/or rraA, or a variant thereof, operably linked to a promoter followed by expressing the nucleic acid of step (a), thereby producing the recombinant polypeptide and either DjlA and/or RraA and in the transformed cell.

As an alternative feature of the invention we present a vector for transforming a host cell comprising a nucleic acid sequence for a recombinant polypeptide and a nucleic acid sequence for either djlA and/or rraA, or a variant thereof, operably linked to a promoter. A transformed host cell comprising a nucleic acid sequence encoding the recombinant polypeptide and either DjlA and/or RraA, or a variant thereof.

Similar sequences to the E. coli DjlA and RraA are found in other hosts and therefore the invention includes such sequences for the uses described herein. DjlA, for example, has homologues in a broad spectrum of Gram-negative bacteria, specific examples of which are Legionella species, Shigella flexneri, Shewanella putrefaciens, Salmonella typhimurium, Vibrio cholerae, Coxiella burnetii, Haemophilus influenza, Yersinia pestis and Yersinia enterocolitica. In addition, it is possible that the sequences not only work in the host from which they derive, but in any of the preferred hosts. Examples are proteins in eukaryotes that have similar domain organization as DjlA, such as Pam18 and Mdj2 in Saccharomyces cerevisiae, which are known to assist protein translocation from the eukaryotic cytosol into mitochondria. It is believed that these sequences will also have utility in recombinant protein expression in S. cerevisiae, but also in other hosts. RraA is an evolutionarily conserved protein with close homologs in bacteria, archaea, proteobacteria, and plants. Examples include proteins from Mycobacterium tuberculosis, Vibrio vulnificus, Thermus thermophiles, Vibrio cholera, and Arabidopsis thaliana.

The invention relates to a method of producing a recombinant polypeptide comprising the steps of: (a) providing a nucleic acid comprising a sequence for the recombinant polypeptide and a nucleic acid sequence for either djlA and/or rraA, or a variant thereof, operably linked to a promoter into an expression system; and (b) expressing the nucleic acid sequences of step (a) under conditions that allow expression of the polypeptide, thereby producing a recombinant polypeptide and either DjlA and/or RraA and, optionally, purifying the recombinant polypeptide.

The invention relates to a method of transforming a host cell with (a) a nucleic acid comprising a sequence for a recombinant polypeptide and a sequence for either djlA and/or rraA operably linked to a promoter followed by expressing the nucleic acid of step (a), thereby producing the recombinant polypeptide and either DjlA and/or RraA and in the transformed cell.

The invention relates to a vector for transforming a host cell comprising a nucleic acid sequence for a recombinant polypeptide and a nucleic acid sequence for either djlA and/or rraA operably linked to a promoter.

The invention relates to a transformed host cell comprising a nucleic acid sequence of the invention, e.g., a sequence encoding the recombinant polypeptide and either djlA and/or rraA.

The invention relates to a modified host cell overexpressing djlA and/or rraA or variants thereof that produce elevated levels of DjlA and/or RrrA or variants thereof.

The invention relates to a modified host cell expressing improved djlA and/or rraA mutants or improved DjlA and/or RraA or variants.

The invention relates to a modified host cell expressing mutants of the gene me encoding for variants of the ribonuclease RNase E with depleted ribonucleolytic activity.

DjlA is a membrane-bound DnaK co-chaperone containing a C-terminal J domain, which is essential for the interaction with DnaK. Apart from DjlA, there are two additional DnaK co-chaperones in E. coli, DnaJ and CbpA. Furthermore, the E. coli genome encodes for three additional J domain proteins (JDPs), HscB (Hsc20), DjlB, and DjlC (HscD or Hsc56), which are not known to interact with DnaK. Instead, HscB forms together with HscA a chaperone/co-chaperone complex similar to DnaK/DnaJ, which is strictly dedicated to the assembly of Fe—S cluster proteins, DjlB is predicted to be membrane-bound and of unknown function, while DjlC is also membrane-bound and a co-chaperone of the E. coli Hsp70 HscC, which shows ATPase activity, but has not been shown to show chaperone activity. Very interestingly, however, no other JDP was found to act as a suppressor of BR2-induced toxicity or as an enhancer of BR2 production (FIGS. 4B and C). This suggests that DjlA is unique among its analogues in its ability to facilitate bacterial recombinant protein production. DjlA overexpression is known to induce a stress response, the Rcs response, and stimulate colanic acid production. Here, we have shown that this response is not involved in the improved phenotype of MP overexpression, as this phenotype is maintained in the rcsC and rcsB⁻ mutants. Without these factors, the Rcs pathway cannot be activated. This response is induced upon djlA overexpression from a high-copy number vector. However, it has been shown that the Rcs response can even be activated by a 2-fold increase in DjlA levels and it is therefore possible that this response is activated in our experiments.

Suppression of BR2-induced toxicity by DjlA and RraA was found to be independent of the particular E. coli strain selected as the target MP production host, as both factors were found to be efficient in MC1061, MC4100A, BW25113 (E. coli K-12 strains) as well as BL21(DE3) (E. coli B strain).

Co-expression of djlA or rraA also resulted in a large increase in the individual cell fluorescence of E. coli cells producing BR2-GFP (FIG. 2D). This suggests that the identified effector genes may also increase the capacity of the bacterial cell to produce BR2 protein, despite the fact that they were not directly selected for this property.

RraA is a protein that acts as a regulator of the mRNA-degrading activity of RNase E and rraA overexpression has been found to globally increase the levels of more than 2,000 different mRNAs in the E. coli cytoplasm. Quantitative real-time PCR analysis, however, revealed that rraA co-expression did not affect BR2 mRNA levels (FIG. 4D), thus demonstrating that the beneficial effects of RraA on recombinant MP production do not occur due to interference with the degradation/stability of the mRNA of the target recombinant protein. Besides, increase of the mRNA levels can even be problematic for the overexpression of MPs as it possibly leads to saturation of the translocon. The RNA abundance is altered globally by rraA overexpression and therefore the mechanism of the downstream membrane biogenesis remains to be elucidated. In the literature it has been shown by microarray analysis that overexpression of RraA altered the mRNA levels of factors involved in anaerobic metabolism and also in cell envelope biosynthesis. Apart from RraA, the E. coli genome encodes for a second similar inhibitor of RNase E, termed RraB, which however affects the RNase E-mediated decay of a different set of transcripts than RraA. Consistently with this, rraB co-expression was found ineffective in suppressing BR2 toxicity and enhancing BR2 accumulation, although it is overexpressed successfully (FIGS. 4A, E and F). Its apparent molecular weight is higher than the theoretical molecular weight calculated in our experiments as has previously been reported. Thus, it is demonstrated again that the identified effector is unique among its analogues in the ability to assist recombinant MP production.

Whilst not wishing to be bound by theory, we believe that the effect of djlA and rraA co-expression improves the membrane integrity. We found that djlA and rraA co-expression resulted in a marked decrease in the extent that BR2-overexpressing cells could be stained with propidium iodide, thus indicating that DjlA and RraA assist the bacterial cell membrane to maintain its integrity during the otherwise toxic process of producing BR2 (FIG. 1J). BR2 overexpression compromises the permeability of the bacterial cytoplasmic membrane, while the integrity of the BR2-overexpressing cells upon djlA and rraA co-expression was restored. As the integrity of the membrane is indicative of the health of the cells the physiology of the cells is restored when the effectors are co-expressed potentially due to reduction of stress.

### BRIEF DESCRIPTION OF THE FIGURES

FIG. 1: shows (A) schematic representation of the utilized BR2-GFP fusion, where GFP is attached to the C-terminal tail of BR2, which is expected to be localized in the bacterial cytoplasm. FLAG and hexa-histidine tags have added to the N and C termini of the fusion protein, respectively. (A). (Top) E. coli MC1061 cells carrying the vector pBAD30BR2-GFP and grown on agar plates without (X) and with ( [symbol] inducer of BR2-GFP production. (Bottom) E. coli MC1061 cells producing BR2-GFP as above, while simultaneously overexpressing djlA (10 μM IPTG), rraA (100 μM IPTG), or a randomly selected gene from the ASKA library (later on determined to be yghS) at 25° C. for 3 days. (B). Effect of djlA (0.01% L(+)-arabinose) and rraA (0.2% L(+)-arabinose) co-expression from pBAD33 on the growth of E. coli MC1061 cells in the presence (left) and absence (right) of BR2-GFP overexpression from pASKBR2-GFP (0.2 μg/ml anhydrotetracycline, aTc) for 16 h at 25° C. OD: optical density. (D). Fluorescence of E. coli MC1061 (WT), SuptoxD and SuptoxR cells producing BR2-GFP as in (C). Bulk fluorescence corresponding to an equal number of cells was measured on a plate reader after overnight induction at 25° C. (left), while levels of individual cell fluorescence were measured by flow cytometry after induction for 4 h at 25° C. (right). M: mean fluorescence. (E). SDS-PAGE/Western blot analysis using an anti-GFP antibody on clarified lysates of E. coli MC1061, SuptoxD and SuptoxR cells producing BR2-GFP as in (C). An equal number of cells were loaded in each lane. MW: molecular weight. (F). Growth of E. coli MC1061, SuptoxD and SuptoxR cells producing FLAG-BR2 from pASKFLAG-BR2 as in (C). OD: optical density. (G). SDS-PAGE/Western blot analysis using an anti-FLAG antibody (top) on isolated total membranes of E. coli MC1061, SuptoxD and SuptoxR cells producing FLAG-BR2 as in (F). Each lane corresponds to a sample of total membranes isolated from an equal number of cells, as verified by using an antibody against the E. coli maltose-binding protein (MBP) (bottom). Four-fold (4×) and eight-fold (8×) more total membrane preparation was loaded for better visualization of FLAG-BR2 accumulation in WT cells. (H). Fluorescence of spheroplasted E. coli MC1061, SuptoxD and SuptoxR cells producing FLAG-BR2 as in (F) and labeled with an Alexa Fluor 647-conjugated anti-FLAG antibody. Spheroplasted bacterial cells overexpressing an N-terminally FLAG-tagged cytoplasmic protein (the DNA-binding domain of human p53) were used as a negative control. Measurements were carried out on a plate reader and correspond to an equal number of cells. (I). Fluorescence of E. coli MC1061 (WT), SuptoxD and SuptoxR cells producing BR2-GFP as in (C). Measurements correspond to cells derived from an equal volume of bacterial culture for each strain. In all measurements of relative fluorescence, the fluorescence of BR2-producing MC1061 cells was arbitrarily set to one. (I). Propidium iodide staining of E. coli MC1061 cells overexpressing BR2 in the absence (pBAD33) or presence of DjlA or RraA co-expression. In all panels, experiments were carried out in replica triplicates and the error bars represent one standard deviation from the mean value.

FIG. 2: SuptoxD and SuptoxR broadly enhance recombinant production for a variety of homologous and heterologous MPs. (A). Growth of E. coli MC1061, SuptoxD and SuptoxR cells producing either NKR1-GFP or CB2-GFP from pASKNKR1-GFP or pASKCB2-GFP, respectively, by the addition of 0.2 μg/ml aTc overnight at 25° C. OD: optical density. (B). Fluorescence of an equal number of E. coli MC1061, SuptoxD and SuptoxR cells producing either NKR1-GFP or CB2-GFP as in (A). The fluorescence of NKR1-producing MC1061 cells was arbitrarily set to one. (C). Fluorescence of equal culture volumes of E. coli MC1061, SuptoxD and SuptoxR cells producing different MP-GFP fusions from the pASK75 vector as in (A). For visualization purposes, the relative fluorescence values for CB2, YidC, MdfA, GsiC were multiplied by four and the relative fluorescence values of ArtM were multiplied by ten. The fluorescence of MC1061 cells producing BR2-GFP was arbitrarily set to one. (D). Comparison of the in-gel fluorescence of total membrane preparations of equal culture volumes of MC1061 (WT) and SuptoxD or SuptoxR cells producing BR2-, NTR1(D03)-, or SapC-EGFP. (E). Growth of E. coli MC1061, SuptoxD and SuptoxR cells producing NTR1(D03)-TrxA from pASKNTRI(D03)-TrxA as in (A). OD: optical density. (F). Fluorescence of BODIPY-NT(8-13)-labelled E. coli MC1061, SuptoxD and SuptoxR spheroplasts producing NTR1(D03)-TrxA from pASKNTRI(D03)-TrxA as in (D) and measured by flow cytometry. The fluorescence of BODIPY-NT(8-13)-labelled E. coli MC1061 cells was arbitrarily set to one. (G). SDS-PAGE/Western blot analysis using an anti-FLAG antibody (top) on isolated total membranes of E. coli MC1061, SuptoxD and SuptoxR cells producing NTR1(D03)-TrxA from pASKNTRI(D03)-TrxA as in (A). Each lane corresponds to a sample of total isolated membranes derived from an equal number of cells, as verified by utilizing an anti-MBP antibody (bottom). Four-fold (4×) and eight-fold (8×) more total membrane preparation was loaded for better visualization of NTR1(D03)-TrxA accumulation in WT cells. MW: molecular weight. In all panels, experiments were carried out in replica triplicates and the error bars represent one standard deviation from the mean value.

FIG. 3: Comparison of the MP production capabilities of SuptoxD and SuptoxR cells with commercial strains frequently utilized for MP production purposes. Fluorescence of MC1061, SuptoxD, and SuptoxR cells producing BR2, CB2, MotA, or SapC, as C-terminal GFP fusions from the pASKBR2-GFP (A), pASKCB2-GFP (B), pASKMotA-GFP (C), and pASKSapC-GFP (D) vectors, respectively, by the addition of 0.2 μg/ml aTc overnight at 25° C., with the fluorescence of equal culture volumes of C41(DE3), C43(DE3), and Lemo21(DE3) cells producing the same proteins from the pETBR2-GFP, pETCB2-GFP, pETMotA-GFP, and pETSapC-GFP vectors, respectively, by the addition of 0.4 mM IPTG and optimal L-rhamnose concentrations (as determined in Supplementary Figure S7) overnight at 25° C. In all cases, the fluorescence of the strain exhibiting the highest level of MP accumulation for each MP-GFP fusion was arbitrarily set to ten. Experiments were carried out in replica triplicates and the error bars represent one standard deviation from the mean value.

FIG. 4: DjlA and RraA are unique among their analogs in their ability to overcome MP-induced cytotoxicity and enhance recombinant MP accumulation. (A). SDS-PAGE/Western blot of total lysates of E. coli MC1061 cells overexpressing djlA, dnaJ, cbpA, hscB, rraA or rraB from pBAD33 in the presence of 0.2% L(+)-arabinose for 16 h at 25° C. and probed with an anti-polyHis antibody. (B). Growth of E. coli MC1061 cells producing BR2-GFP from pASKBR2-GFP by the addition of 0.2 μg/ml aTc overnight at 25° C., without/with co-expression of djlA and all other E. coli JDP-encoding genes (0.01% L(+)-arabinose). OD: optical density. (C). Fluorescence of E. coli MC1061 cells producing BR2-GFP without/with co-expression of djlA and all other E. coli JDP-encoding genes as in (B). Measurements correspond to an equal number of cells. The fluorescence of cells producing BR2-GFP in the absence of effector co-expression was arbitrarily set to one. (D). Comparison of BDKRB2 mRNA levels without/with co-expression of rraA using real-time PCR. Two independent experiments were carried out in replica triplicates and the error bars represent one standard deviation from the mean value (E). Growth of E. coli MC1061 cells producing BR2-GFP as in (B) without/with co-expression of rraA or rraB (0.2% L(+)-arabinose). (F). Fluorescence of E. coli MC1061 cells producing BR2-GFP without/with co-expression of rraA or rraB as in (E). Measurements correspond to an equal number of cells. The fluorescence of E. coli MC1061 cells producing BR2-GFP was arbitrarily set to one. (G). Growth of E. coli BW25113 djlA⁻ or rraA⁻ cells producing BR2-GFP as in (B) without/with co-expression of djlA or rraA. (H). Fluorescence of E. coli BW25113 djlA⁻ or rraA⁻ cells producing BR2-GFP without/with co-expression of djlA or rraA as in (G). Measurements correspond to an equal number of cells. The fluorescence of E. coli BW25113 cells producing BR2-GFP was arbitrarily set to one. In all panels, experiments were carried out in replica triplicates and the error bars represent one standard deviation from the mean value unless otherwise stated.

FIG. 5: Full-length, membrane-bound DjlA that is capable of interacting with DnaK is necessary for its beneficial effects on MP production. (A). Schematic representation of the functional domain organization of DjlA, DnaJ, and the tested DjlA variants. DjlA is composed of an N-terminal transmembrane domain (TMD, dark grey), a central domain of unknown function (central, grey) and a C-terminal J domain that contains the conserved HPD motif (J, green). DnaJ is composed of an N-terminal J domain (J, green), a glycine/phenylalanine-rich domain (G/F, red), a zinc-binding domain (Zn, blue) and a C-terminal domain (CTD, darkest grey). (B). Sequence alignment of the J domains of DjlA and DnaJ. Identical amino acids are highlighted in yellow and similar ones are highlighted in green. (C). SDS-PAGE/Western blot analysis of total lysates of E. coli MC1061 cells overexpressing djlA and the tested djlA mutants, probed with an anti-polyHis antibody. Seven-fold (7×) more lysate was loaded for DjlAΔcentral to allow visualization. (D). Growth of E. coli BW25113 djlA cells producing BR2-GFP from pASKBR2-GFP by the addition of 0.2 μg/ml aTc overnight at 25° C. without/with co-expression of djlA and the tested djlA mutants (0.01% L(+)-arabinose). OD: optical density. The reported values correspond to the mean fluorescence of an equal number of cells from three independent experiments performed in triplicate and the presented error bars to one standard deviation from the mean value. (E). Fluorescence of E. coli BW25113 djlA⁻ cells producing BR2-GFP without/with co-expression of djlA and the tested djlA mutants as in (D). The fluorescence of cells producing BR2-GFP in the absence of effector co-expression was arbitrarily set to one. The reported values correspond to the mean fluorescence of an equal number of cells from three independent experiments performed in triplicate and the presented error bars to one standard deviation from the mean value. (F). Growth of E. coli W3110 or W3110 dnaK⁻ cells producing BR2-GFP from the plasmid pASK(KanR)BR2-GFP without/with djlA co-expression from the plasmid pBAD30DjlA as in (D). (G). Fluorescence of equal culture volumes of E. coli W3110 or W3110 dnaK⁻ cells producing BR2-GFP from the plasmid pASK(KanR)BR2-GFP without/with djlA co-expression from the plasmid pBAD30DjlA as in (D). The fluorescence of W3110 cells producing BR2-GFP in the absence of effector co-expression was arbitrarily set to one. (H). Growth of E. coli BW25113, BW25113 rcsB⁻, or BW25113 rcsC⁻ cells producing BR2-GFP with and without djlA co-expression as in (D). (I). Fluorescence of E. coli BW25113, BW25113 rcsB-, or BW25113 rcsC⁻ cells producing BR2-GFP without/with djlA co-expression as in (D). The fluorescence of BW25113 cells producing BR2-GFP in the absence of effector co-expression was arbitrarily set to one. In all cases, fluorescence measurements correspond to an equal number of cells unless otherwise stated. In all panels, experiments were carried out in replica triplicates and the error bars represent one standard deviation from the mean value unless otherwise stated.

FIG. 6: The beneficial effects of RraA on recombinant MP production in E. coli are mediated by the ribonuclease RNase E, but not its paralogous protein RNase G. (A). (top) Schematic representation of the domain organization of the E. coli RNase E and of the different RNase E variants expressed by the E. coli me mutants utilized in this study. The catalytic domain of RNase E is located at the N terminus of the protein (residues 1-529). The interaction of other components of the degradosome with RNase E takes place at its C-terminal domain (CTD). The microdomains of the CTD are color-coded; white indicates intrinsically disordered regions; red indicates the membrane targeting sequence (MTS; residues 565-582); green indicates the arginine-rich regions 1 and 2 (AR1, AR2; residues 604-644 and 796-814, respectively); light blue indicates the helicase binding site (HBS; residues 719-731); purple indicates the enolase-binding site (EBS; residues 834-850) and orange indicates the PNPase-binding site (PBS; residues 1021-1061). Known RraA interaction sites are indicated by grey arrows. (bottom) Schematic representation of the domain organization of the E. coli RNase E paralogous protein, RNase G. The N-terminal catalytic region of RNase G has a high sequence homology to the catalytic domain of RNase E. The C-terminal region of RNase G, however, lacks regulatory microdomains, such as those contained in RNase E. (B). Growth of E. coli ENS134 (wild-type) or ENS134 me mutant strains expressing wild-type RNase E or truncated forms thereof and producing BR2-GFP from pASKBR2-GFP by the addition of 0.2 μg/ml aTc overnight at 25° C. without/with rraA co-expression (0.2% L(+)-arabinose). The reported values correspond to the mean value from n=3 independent experiments, each one performed in triplicate±s.e.m. Statistical significance is denoted as follows: *P≤0.05, **P≤0.01, not significant>0.05 and corresponds to the differences between the [+RraA] vs. the [no effector] samples for each strain. (C). Fluorescence of an equal number of cells derived from E. coli ENS134 (wild-type) or ENS134 me mutant strains producing BR2-GFP as in (b). Values and errors are reported as in (b). (D). Fluorescence of equal culture volumes derived from E. coli ENS134 (wild-type) or ENS134 me mutant strains producing BR2-GFP as in (b). Values and errors are reported as in (b). (E). Growth (left) and fluorescence (right) of E. coli BW25113 or BW25113 rng cells producing BR2-GFP without/with rraA co-expression as in (b). (F). Growth (left) and fluorescence (right) of E. coli MC1061 cells producing BR2-GFP as in (b) without/with co-expression of rraA or rraB (0.2% L(+)-arabinose). (G). (left) SDS-PAGE/western blot analysis using anti-FLAG (left) and anti-polyHis (right) antibodies on isolated total membranes of E. coli MC1061 cells producing BR2-GFP without/with co-expression of rraA or rraB as in (f). Each lane corresponds to a sample of total membranes isolated from an equal volume of bacterial culture. (right) SDS-PAGE/western blot analysis of total lysates of E. coli MC1061 cells overexpressing rraA or rraB from pBAD33RraA and pBAD33RraB, respectively, in the presence of 0.2% L(+)-arabinose for 16 h at 25° C. and probed with an anti-polyHis antibody. Ten-fold more cell lysate was loaded on the gel in the case of rraB overexpression compared to rraA. (h). Growth of E. coli MC1061 cells producing CB1-GFP (left), CB2-GFP (middle) or TcyL-GFP (right) from pASKCB1-GFP, pASKCB2-GFP and pASKTcyL-GFP, respectively, by the addition of 0.2 μg/ml aTc overnight at 25° C. without/with co-expression of rraA or rraB as in (f). (i). Fluorescence of equal culture volumes of E. coli MC1061 cells producing CB1-GFP (left), CB2-GFP (middle) or TcyL-GFP (right) without/with co-expression of rraA or rraB as in (h). In (e), (f), (h) and (i), experiments were carried out in replica triplicates and the error bars represent one standard deviation from the mean value. The fluorescence of cells producing MP-GFP fusions in the absence of effector co-expression was arbitrarily set to one.

### DETAILED DESCRIPTION OF THE INVENTION

### Variants of DjlA and RraA

“Variants” are functional variants of DjlA and RraA and are anticipated to work as well or even better, or not significantly any worse, than DjlA and RraA themselves. Variants can be produced by genetic or protein engineering, such as by the use of the following standard techniques. “Variants” include silent changes the nucleotide sequence that do not change the amino acid sequence expressed.

DjlA and RraA were first discovered as fusions with a C-terminal polyhistidine tag. We have found that they work both in tagged and untagged form. Therefore, within the definition of “variant”, we include C-terminal polyhistidine tagged and C-terminal polyhistidine untagged sequences.

The term “variant” additional or alternatively includes those nucleic acids which are in essence equivalent to the original nucleic acids encoding DjlA and RraA but showing at least about 40% amino acid identity and/or similarity, more typically at least about 60% or 80%, 90%, 95% and 98% sequence identity and/or similarity to the original DjlA and RraA encoding sequences.

“Variant” additionally or alternatively includes changes which contains, for example, deletions, insertions and/or substitutions in the polynucleotide and/or polypeptide sequence. For example, such changes in the nucleic acid sequence are considered to cause a substitution with an equivalent amino acid. Preferable are such amino acid substitutions that result in substitutions, which substitute one amino acid with a similar amino acid with similar structural and/or chemical properties, i.e. conservative amino acid substitutions. Amino acid substitutions can be performed on the basis of similarity in polarity, charges, solubility, hydrophobic, hydrophilic, and/or amphiphilic nature of the involved residues. Examples for hydrophobic amino acids are alanine, leucine, isoleucine, valine, proline, phenylalanine, tryptophan and methionine. Polar, neutral amino acids include glycine, serine, threonine, cysteine, tyrosine, asparagine and glutamine. Positively (basic) charged amino acids include arginine, lysine and histidine. And negatively charged amino acids include aspartic acid and glutamic acid. “Insertions” or “deletions” usually range from one to several hundred amino acids. The allowed degree of variation can be experimentally determined via methodically applied insertions, deletions or substitutions of amino acids in a protein molecule using recombinant DNA methods. The resulting variants can be tested for their biological activity. Examples include, the nucleic acid sequences being mutagenized using conventional techniques, such as site-directed mutagenesis, or other techniques familiar to those skilled in the art, to introduce silent changes into the nucleotide sequences. Such changes may be desirable in order to increase the level of the polypeptide produced by host cells containing a vector encoding the polypeptide by introducing codons or codon pairs, which occur frequently in the host organism.

The term “variant” additionally or alternatively includes polynucleotide sequences which have nucleotide changes which result in amino acid substitutions, additions, deletions, fusions and truncations in the polypeptides of DjlA and RraA. Such nucleotide changes may be introduced using techniques such as site directed mutagenesis, random chemical mutagenesis, exonuclease III deletion and other recombinant DNA techniques.

Alternatively and additionally “variants” also includes such nucleotide changes that may be naturally occurring in allelic variants which are isolated by identifying nucleic acids which specifically hybridize to probes comprising at least 10, 15, 20, 25, 30, 35, 40, 50, 75, 100, 150, 200, 300, 400, or 500 consecutive bases of one of the sequences of. The invention and sequences substantially identical thereto (or the sequences complementary thereto) under conditions of high, moderate, or low stringency.

Homology may be determined using any of the computer programs and parameters described herein, including FASTA version 3.0t78 with the default parameters or with any modified parameters. The homologous sequences may be obtained using any of the procedures described herein or may result from the correction of a sequencing error. The polypeptide fragments comprise at least about 10, 15, 20, 25, 30, 35, 40, 45, 50, 75, 100 homology to the sequences.

In alternative embodiments, a polynucleic acid sequence of the invention can be altered by any means to create a variant. For example, random or stochastic methods, or, non-stochastic, or “directed evolution,” methods, see, e.g., U.S. Pat. No. 6,361,974. Methods for random mutation of genes are well known in the art, see, e.g., U.S. Pat. No. 5,830,696. For example, mutagens can be used to randomly mutate a gene. Mutagens include, e.g., ultraviolet light or gamma irradiation, or a chemical mutagen, e.g., mitomycin, nitrous acid, photoactivated psoralens, alone or in combination, to induce DNA breaks amenable to repair by recombination. Other chemical mutagens include, for example, sodium bisulfite, nitrous acid, hydroxylamine, hydrazine or formic acid. Other mutagens are analogues of nucleotide precursors, e.g., nitrosoguanidine, 5-bromouracil, 2-aminopurine, or acridine. These agents can be added to a PCR reaction in place of the nucleotide precursor thereby mutating the sequence. Intercalating agents such as proflavine, acriflavine, quinacrine and the like can also be used. Any technique in molecular biology can be used, e.g., random PCR mutagenesis, see, e.g., Rice (1992) Proc. Natl. Acad. Sci. USA 89:5467-5471; or, combinatorial multiple cassette mutagenesis, see, e.g., Crameri (1995) Biotechniques 18:194-196. Alternatively, nucleic acids, e.g., genes, can be reassembled after random, or “stochastic,” fragmentation, see, e.g., U.S. Pat. Nos. 6,291,242; 6,287,862; 6,287,861; 5,955,358; 5,830,721; 5,824,514; 5,811,238; 5,605,793. In alternative aspects, modifications, additions or deletions are introduced by error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly (e.g., GeneReassembly, see, e.g., U.S. Pat. No. 6,537,776), gene site saturation mutagenesis (GSSM), synthetic ligation reassembly (SLR), recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, and/or a combination of these and other methods.

The following publications describe a variety of recursive recombination procedures and/or methods which can be incorporated into the methods of the invention: Stemmer (1999) “Molecular breeding of viruses for targeting and other clinical properties” Tumor Targeting 4:1-4; Ness (1999) Nature Biotechnology 17:893-896; Chang (1999) “Evolution of a cytokine using DNA family shuffling” Nature Biotechnology 17:793-797; Minshull (1999) “Protein evolution by molecular breeding” Current Opinion in Chemical Biology 3:284-290; Christians (1999) “Directed evolution of thymidine kinase for AZT phosphorylation using DNA family shuffling” Nature Biotechnology 17:259-264; Crameri (1998) “DNA shuffling of a family of genes from diverse species accelerates directed evolution” Nature 391:288-291; Crameri (1997) “Molecular evolution of an arsenate detoxification pathway by DNA shuffling,” Nature Biotechnology 15:436-438; Zhang (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York, pp. 447-457; Crameri and Stemmer (1995) “Combinatorial multiple cassette mutagenesis creates all the permutations of mutant and wildtype cassettes” BioTechniques 18:194-195; Stemmer et al. (1995) “Single-step assembly of a gene and entire plasmid form large numbers of oligodeoxyribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. ScL US A 91: 10747-10751.

Homologous proteins of DjlA and RraA from other organisms exist and these are additionally or alternatively within the definition of “variant”. DjlA, for example, has homologues in a broad spectrum of Gram-negative bacteria, specific examples of which are Legionella species, Shigella flexneri, Shewanella putrefaciens, Salmonella typhimurium, Vibrio cholerae, Coxiella burnetii, Haemophilus influenza, Yersinia pestis and Yersinia enterocolitica. RraA is an evolutionarily conserved protein with close homologs in bacteria, archaea, proteobacteria, and plants. Examples include proteins from Mycobacterium tuberculosis, Vibrio vulnificus, Thermus thermophiles, Vibrio cholera, and Arabidopsis thaliana.

We have found that certain domains of DjlA and RraA are critical and these domains of DjlA and RraA are also additionally or alternatively within the definition of “variant”.

Therefore, specifically the invention can use

| Variant | Toxicity | Expression levels |
|---|---|---|
| DjlA | +++ | +++ |
| DjlAΔTM | − | + |
| DjlAΔJ | − | − |
| DjlAΔcentral | − | +++ |
| DjlAJDnaJ | − | + |
| DjlA(H233Q) | − | − |
| Djla(M16R) | + | + |

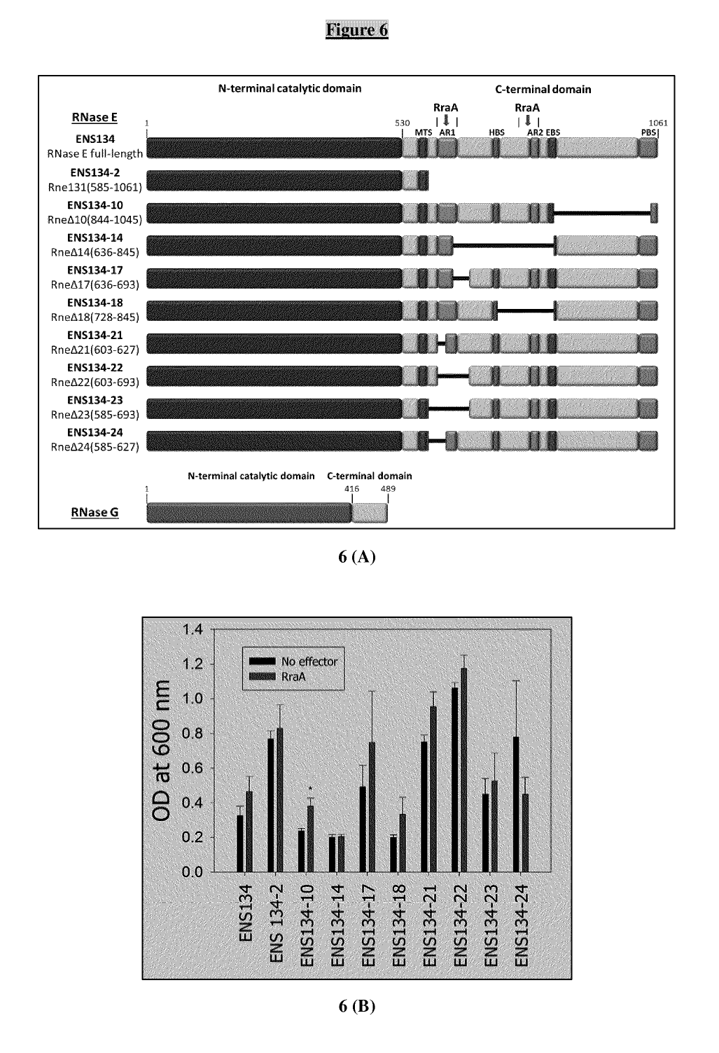

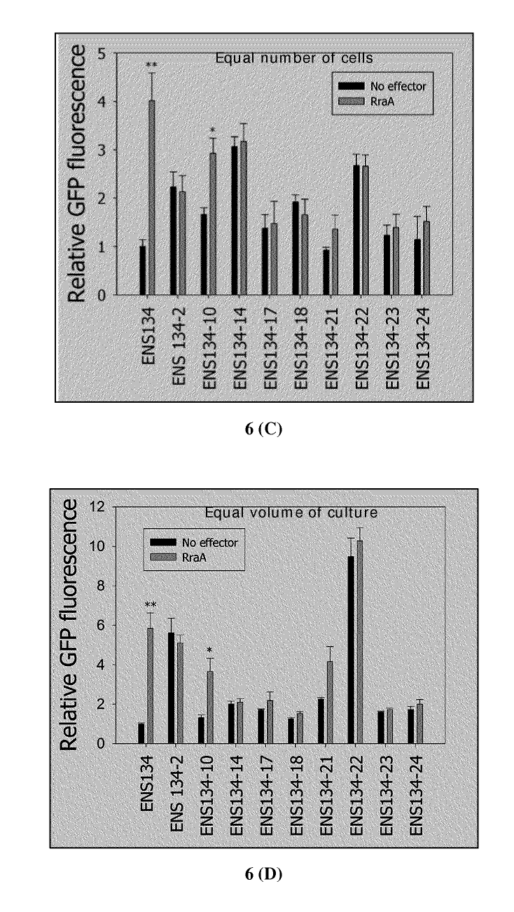

The domain organization of these variants is described in FIG. 5A.

The nucleic acid sequence which encodes one or more of the polypeptides of the invention and sequences substantially identical thereto, may include, but is not limited to: only the coding sequence of a nucleic acid of the invention and sequences substantially identical thereto and additional coding sequences, such as leader sequences or proprotein sequences and non-coding sequences, such as introns or non-coding sequences 5′ and/or 3′ of the coding sequence. Thus, as used herein, the term “nucleic acid sequence encoding a polypeptide or DjlA and RraA” encompasses a polynucleotide which includes only the coding sequence for the polypeptide as well as a polynucleotide which includes additional coding and/or non-coding sequences.

### Expression Vectors

A wide variety of expression vectors for the transformation of the host are anticipated and the type of vectors and sequences therein are dependent upon the host system.

Therefore, a feature of the invention is a vector for the transformation of a cellular host with the expression vector comprising the sequence for either one or both djlA and rraA, or a functional variant thereof, and a promoter sequence. The cellular host is prokaryotic or eukaryotic and includes bacteria, yeast, animal, fungal, animal, human, or plant cells.

Preferred examples of prokaryotic hosts are Escherichia coli, Lactococcus lactis, Bacillus subtilis, Pseudomonas aeruginosa, Erwinia carotovora, Salmonella choleraesuis, Agrobacterium tumefaciens, Chromobacterium violaceum, Salmonella. Preferred examples of eukaryotic hosts are Saccharomyces cerevisae, Pichia pastoris, Schizosaccharomyces pombe, Kluyveromyces lactis, CHO, NS0, HEK293, HeLa, Sf9, tobacco, rice, and Leishmania tarentolae.

The vector includes expression cassettes comprising a nucleic acid comprising a sequence of the invention. The vector can comprise a viral vector, a plasmid, a phage, a phagemid, a cosmid, a fosmid, a bacteriophage or an artificial chromosome. The viral vector can comprise an adenovirus vector, a retroviral vector or an adeno-associated viral vector. The vector can comprise a bacterial artificial chromosome (BAC), a bacteriophage P1-derived vector (PAC), a yeast artificial chromosome (YAC), or a mammalian artificial chromosome (MAC). The vector or overexpression cassette of the effector genes djlA and/or rraA, or variants thereof, can be integrated into the genome of the expression host. The invention provides expression cassettes that can be expressed in a tissue-specific manner such as in plants, insects or other animals (preferably non-human).

Vectors are commercially available and typically comprise one or more of the following, a control sequence(s) and a selectable marker sequence. In the absence of selective pressure plasmids are lost from the host. Preferably the selective marker is an antibiotic-resistance coding sequence and supplement the medium with the appropriate antibiotic to kill. Examples of such antibiotics are ampicillin, carbenicillin, chloramphenicol, kanamycin, rifampicin, and tetracycline. Selectable markers include genes encoding dihydrofolate reductase or genes conferring neomycin resistance for eukaryotic cell culture and the S. cerevisiae TRP1 gene. Selectable markers can also include biosynthetic genes, such as those in the histidine, tryptophan and leucine biosynthetic pathways.

The nucleic acid sequences that are wished to be expressed in the vectors should be operatively linked to one or more control sequences. The control sequence(s) comprises one or more of the following

a. A “promotor” initiates transcription and is positioned 10-100 nucleotides upstream of the ribosome-binding site. The ideal promoter exhibits several desirable features:
It is strong enough to allow product accumulation up to 50% of the total cellular protein.
It has a low basal expression level (i.e. it is tightly regulated to prevent product toxicity).
It is easy to induce.

Examples of typical promoter sequences for E. coli and the associated induction are:

| Name | Induction |
|---|---|
| lac | IPTG |
| tac (hybrid) | IPTG |
| trc (hybrid) | IPTG |
| P_{syn} (synthetic) | IPTG |
| Trp | Tryptophan starvation |
| araBAD | L-arabinose |
| rhaBAD | L-rhamnose |
| lpp | IPTG, lactose |
| lpp-lac (hybrid) | IPTG |
| phoA | Phosphate starvation |
| recA | Nalidixic acid |
| proU | Osmolarity |
| cst-1 | Glucose starvation |
| tetA | Tetracycline |
| cadA | pH |

Eukaryotic promoters include the GAL1 galactose-inducible promoter, the CUP1 copper-inducible promoter, and the MET25 methionine-repressible promoter for yeasts; the CMV immediate early promoter, the HSV thymidine kinase promoter, heat shock promoters, the early and late SV40 promoter, LTRs from retroviruses, and the mouse metallothionein-I promoter. Other promoters known to control expression of genes in prokaryotic or eukaryotic cells or their viruses may also be used.

### Tissue-Specific Plant Promoters

In one aspect, a constitutive promoter such as the CaMV 35S promoter can be used for expression in specific parts of the plant or seed or throughout the plant. For example, for overexpression, a plant promoter fragment can be employed, which will direct expression of a nucleic acid in some or all tissues of a plant, e.g., a regenerated plant. Such promoters are constitutive promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumefaciens, and other transcription initiation regions from various plant genes known to those of skill. Such genes include, e.g., ACTI1 from Arabidopsis (Huang (1996) Plant Mol. Biol. 33:125-139); Cat3 from Arabidopsis (GenBank No. U43147, Zhong (1996) Mol. Gen. Genet. 251:196-203); the gene encoding stearoyl-acyl carrier protein desaturase from Brassica napus (Genbank No. X74782, Solocombe (1994) Plant Physiol. 104:1167-1176); GPcI from maize (GenBank No. X15596; Martinez (1989) J Mol. Biol 208:551-565); the Gpc2 from maize (GenBank No. U45855, Manjunath (1997) Plant Mol. Biol. 33:97-112); plant promoters described in U.S. Pat. Nos. 4,962,028; 5,633,440.

The invention uses tissue-specific or constitutive promoters derived from viruses which can include, e.g., the tobamovirus subgenomic promoter (Kumagai (1995) Proc. Natl. Acad. Sci. USA 92: 1679-1683; the rice tungro bacilliform virus (RTBV), which replicates only in phloem cells in infected rice plants, with its promoter which drives strong phloem-specific reporter gene expression; the cassava vein mosaic virus (CVMV) promoter, with highest activity in vascular elements, in leaf mesophyll cells, and in root tips (Verdaguer (1996) Plant MoL Biol. 31:1129-1139).

b. A regulatory gene (“repressor”) prevents expression from the vector before induction which can be important when the protein expressed is toxic to the host. The repressor sequence is linked to the promoter sequence used and are known in the art.
c. “Origin of replication”. The origin of replication controls the plasmid copy number.
d. “Transcription terminator”. The transcription terminator reduces unwanted transcription and increases plasmid and mRNA stability.
e. “Enhancers” such as the “Shine-Dalgamo sequence”. The Shine-Dalgamo (SD) sequence is required for translation initiation and is complementary to the 3′-end of the 16S ribosomal RNA. The sequence is a ribosomal binding site in prokaryotic messenger RNA, generally located around 8 bases upstream of the start codon AUG. The RNA sequence helps recruit the ribosome to the mRNA to initiate protein synthesis by aligning the ribosome with the start codon. The efficiency of translation initiation at the start codon depends on the actual sequence. The consensus sequence is: 5′-TAAGGAGG-3′. It is positioned 4-14 nucleotides upstream the start codon with the optimal spacing being 8 nucleotides. To avoid formation of secondary structures (which reduces expression levels) this region should be rich in A residues. In eukaryotic cells enhancers to increase expression levels are cis-acting elements of DNA, usually from about 10 to about 300 bp in length that act on a promoter to increase its transcription. Examples include the SV40 enhancer on the late side of the replication origin bp 100 to 270, the cytomegalovirus early promoter enhancer, the polyoma enhancer on the late side of the replication origin, and the adenovirus enhancers.
f. “Start codon”. Initiation point of translation. In E. coli the most used start codon is ATG. GTG is used in 8% of the cases. TTG and TAA are hardly used.
g. “Stop codon”. Termination of translation. There are 3 possible stop codons for E. coli but TAA is preferred because it is less prone to read-through than TAG and TGA. The efficiency of termination is increased by using 2 or 3 stop codons in series.

Optional additional aspects to include in the vector are one or more the following:

a. “Tags and fusion proteins”. N- or C-terminal fusions of heterologous proteins to short peptides (tags) or to other proteins (fusion partners) offer several potential advantages; (i) improved expression—fusion of the N-terminus of a heterologous protein to the C-terminus of a highly-expressed fusion partner often results in high level expression of the fusion protein, (ii) improved solubility, fusion of the N-terminus of a heterologous protein to the C-terminus of a soluble fusion partner often improves the solubility of the fusion protein (iii) improved detection—fusion of a protein to either terminus of a short peptide (epitope tag) or protein which is recognized by an antibody or a binding protein (Western blot analysis) or by biophysical methods (e.g. GFP by fluorescence) allows for detection of a protein during expression and purification and (iv) improved purification—simple purification schemes have been developed for proteins fused at either end to tags or proteins which bind specifically to affinity resins.
b. “Protease cleavage site”. Protease cleavage sites are often added to be able to remove a tag or fusion partner from the fusion protein after expression. However, cleavage is rarely complete and often additional purification steps are required.
c. “Multiple cloning site”. A series of unique restriction sites that enables the gene or genes of interest to be overexpressed in the host to be inserted into the vector. In general, the sequence is ligated to the desired position in the vector following digestion of the insert and the vector with appropriate restriction endonucleases. Alternatively, blunt ends in both the insert and the vector may be ligated. A variety of cloning techniques are known in the art. Such procedures and others are deemed to be within the scope of those skilled in the art.

The vector can have two replication systems to allow it to be maintained in two organisms, for example in mammalian or insect cells for expression and in a prokaryotic host for cloning and amplification. Furthermore, for integrating expression vectors, the expression vector can contain at least one sequence homologous to the host cell genome. It can contain two homologous sequences which flank the expression construct. The integrating vector can be directed to a specific locus in the host cell by selecting the appropriate homologous sequence for inclusion in the vector. Constructs for integrating vectors are well known in the art.

Particular bacterial vectors which can be used include the commercially available plasmids comprising genetic elements of the well-known cloning vector pBR322 (ATCC 37017), pKK223-3 (Pharmacia Fine Chemicals, Uppsala, Sweden), GEM1 (Promega Biotec, Madison, Wis., USA) pQE70, pQE60, pQE-9 (Qiagen), pDIO, psiX174 pBluescript II KS, pNH8A, pNH16a, pNH18A, pNH46A (Stratagene), ptrc99a, pKK233-3, DR540, pRIT5 (Pharmacia), pKK232-8 and pCM7. Particular eukaryotic vectors include pSV2CAT, pOG44, pXT1, pSG (Stratagene) pSVK3, pBPV, pMSG, and pSVL (Pharmacia). However, any other vector may be used as long as it is replicable and viable in the host cell.

Expression vectors capable of expressing nucleic acids and proteins in plants are well known in the art, and can include, e.g., vectors from Agrobacterium spp., potato virus X (see, e.g., Angell (1997) EMBO J. 16:3675-3684), tobacco mosaic virus (see, e.g., Casper (1996) Gene 173:69-73), tomato bushy stunt virus (see, e.g., Hillman (1989) Virology 169:42-50), tobacco etch virus (see, e.g., Dolja (1997) Virology 234:243-252), bean golden mosaic virus (see, e.g., Morinaga (1993) Microbiol Immunol. 37:471-476), cauliflower mosaic virus (see, e.g., Cecchini (1997) Mol. Plant Microbe Interact. 10:1094-1101), maize Ac/Ds transposable element (see, e.g., Rubin (1997) Mol. Cell. Biol. 17:6294-6302; Kunze (1996) Curr. Top. Microbiol. Immunol. 204:161-194), and the maize suppressor-mutator (Spin) transposable element (see, e.g., Schlappi (1996) Plant MoL Biol. 32:717-725); and derivatives thereof.

The vector can be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis, L., Dibner, M., Battey, I., Basic Methods in Molecular Biology, (1986)).

Expression Systems Other than E. coli

The invention provides transformed cells comprising a nucleic acid or vector of the invention, or an expression cassette or cloning vehicle of the invention. The transformed cell can be a bacterial cell, a mammalian cell, a fungal cell, a yeast cell, an insect cell or a plant cell.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof. Cell free expression systems are described in (FEBS Journal. High level cell-free expression and specific labeling of integral membrane proteins, Christian Klammt, Frank Löhr, Birgit Schäfer, Winfried Haase, Volker Dötsch, Heinz Rüterjans, Clemens Glaubitz, Frank Bernhard, Volume 271, Issue 3, February 2004, Pages 568-580; Membrane protein synthesis in cell-free systems: from bio-mimetic systems to bio-membranes. Sachse R, Dondapati S K, Fenz S F, Schmidt T, Kubick S. FEBS Lett. 2014 Aug. 25; 588(17):2774-81).

Where appropriate, the engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment thereof can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxyapatite chromatography and lectin chromatography. Protein refolding steps can be used, as necessary, in completing configuration of the polypeptide. If desired, high-performance liquid chromatography (HPLC) can be employed for final purification steps.

The host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides produced may or may not also include an initial methionine amino acid residue.

### EXAMPLES

### Example 1: Genetic Screening for the Identification of Factors that Suppress the Cytotoxicity Caused by Membrane Protein Overexpression

As a model membrane protein, we chose the human bradykinin receptor 2 (BR2), a therapeutically relevant G protein-coupled receptor (GPCR). BR2 overexpression in E. coli has been found to be very toxic, resulting in complete growth arrest upon induction of protein production in liquid cultures, despite the fact that it accumulates in membrane-integrated form only at low levels.

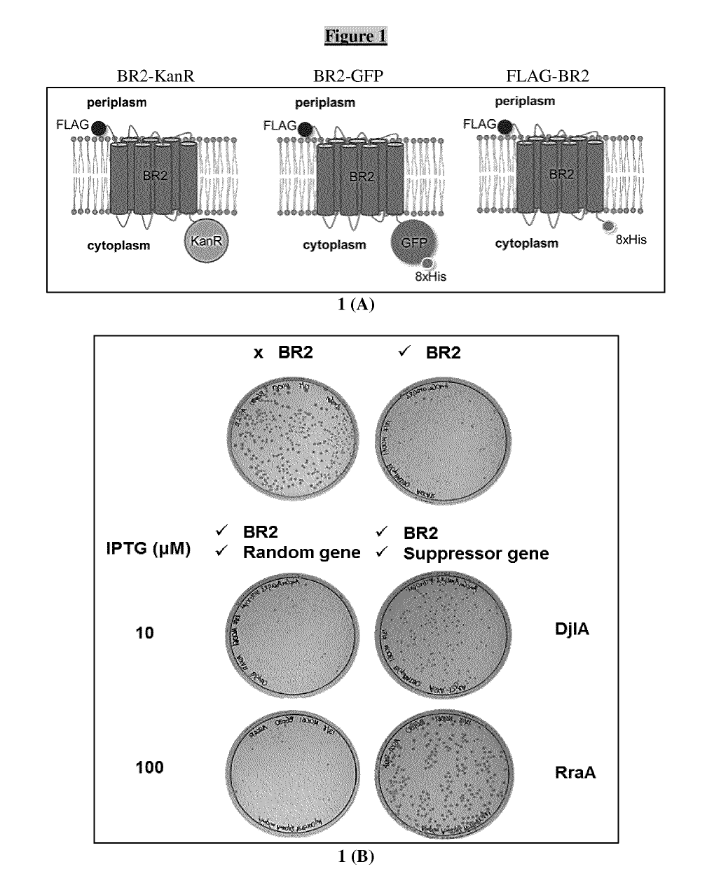

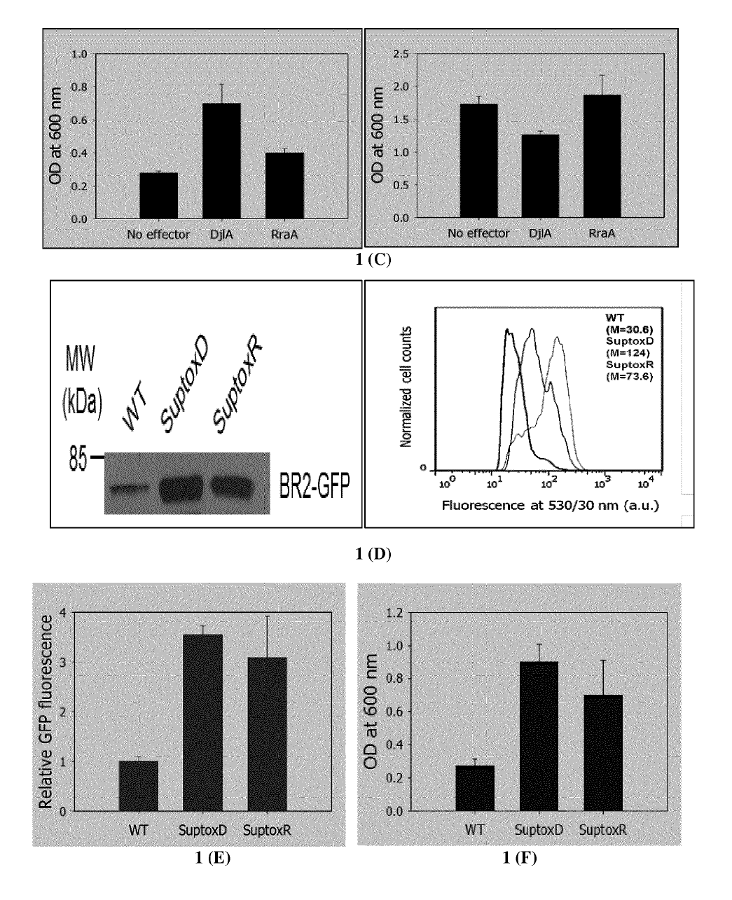

E. coli MC1061 cells (Table 2) were initially transformed with the BR2-encoding vector pBAD30BR2-KanR. pBAD30BR2-KanR expresses a fusion of BR2 with a FLAG tag attached to its N terminus and the sequence of aminoglycoside 3′-phosphotransferase (KanR—the enzyme conferring resistance to the antibiotic kanamycin) followed by a hexahistidine tag attached to its C terminus (Table 3; FIG. 1A). Expression of this fusion protein is under the control of the araBAD promoter. The FLAG and hexahistidine sequences serve as tags for immunodetection and can facilitate protein purification, while KanR allows facile monitoring of BR2 production levels by recording the levels of bacterial growth in the presence of kanamycin. E. coli MC1061 cells carrying pBAD30BR2-KanR were plated onto LB agar plates in the absence and presence of L(+)-arabinose, the inducer of BR2 overexpression, and we observed that the size of the bacterial colonies formed was dramatically decreased when arabinose was present in the medium (FIG. 1B), thus demonstrating that BR2-induced cytotoxicity is also evident when overexpression takes place on solid media. This toxicity phenotype both in liquid media and on agar plates was found to be independent of the presence of the KanR fusion partner.

In order to look for genes that act as suppressors of membrane protein-induced toxicity, electro-competent MC1061 cells carrying pBAD30BR2-KanR were co-transformed with the ASKA (A Complete Set of Escherichia coli K-12 ORF Archive) library, an ordered library of plasmids encoding all known E. coli open reading frames (ORFs) under the control of the T5lac promoter. A total of approximately 120,000 transformants were plated onto three different large LB agar plates containing: (i) ampicillin and chloramphenicol, the appropriate antibiotics to ensure plasmid maintenance, (ii) 0.2% arabinose to induce BR2 production, (iii) a low concentration (10 μg/ml) of kanamycin to ensure that full-length receptor is produced and that gene, plasmid, chromosomal etc. mutations that block BR2 expression do not accumulate, and (iv) three different isopropyl-D-thiogalactopyranoside (IPTG) concentrations to induce overexpression of the ASKA library genes: 0, 0.01, and 0.1 mM. Low to medium concentrations of IPTG were preferred as it has been found that high-level induction from the ASKA plasmids at 1 mM IPTG results in severe growth inhibitions for more than half of all the genes contained in this library. After incubation for two overnights at 30° C., large colonies on a background of pinprick-sized colonies appeared on all three plates, indicating potential suppressed toxicity. 140 such large colonies were picked and their corresponding ASKA vectors were isolated. In order to test whether the loss-of-toxicity phenotype is due to the presence of the gene encoded in the ASKA vectors, fresh MC1061 cells were transformed with the pBAD30BR2-KanR vector and the ASKA plasmids isolated from bacterial cultures inoculated with these large colonies. The growth of the resulting transformants was evaluated in liquid LB cultures under conditions where BR2 production is toxic. For approximately 100 of these clones, increased cell densities were recorded, compared to control cultures carrying randomly selected ASKA plasmids, demonstrating that increased growth occurs indeed due to the presence of the identified ASKA-encoding genes.

TABLE 2
E. coli strains used in this study

| Strain | Genotype | Source |
|---|---|---|
| MC1061 | F⁻ λ⁻ Δ(ara-leu)7697 [araD139]B/r Δ(codB-lacI)3 | Laboratory |
| | galK16 galE15 e14⁻ mcrA0 relA1 rpsL150(Str^{R}) | collection |
| | spoT1 mcrB1 hsdR2(r⁻m⁺) | |
| BL21(DE3) | F⁻ ompT gal dcm lon hsdSB(rB− mB−) λ(DE3 [lacI | Laboratory |
| | lacUV5-T7 gene 1 ind1 sam7 nin5]) | collection |
| BW25113 | F⁻, Δ(araD-araB)567, ΔlacZ4787(::rrnB-3), λ⁻, rph- | Keio |
| | 1, Δ(rhaD-rhaB)568, hsdR514 | collection |
| BW25113 | F⁻, ΔdjlA766::kan, Δ(araD-araB)567, | Keio |
| djlA⁻ | ΔlacZ4787(::rrnB-3), λ⁻, rph-1, Δ(rhaD-rhaB)568, | collection |
| | hsdR514 | |
| BW25113 | F⁻, Δ(araD-araB)567, ΔlacZ4787(::rrnB-3), λ⁻, rph- | Keio |
| rraA⁻ | 1, Δ(rhaD-rhaB)568, ΔrraA788::kan, hsdR514 | collection |
| BW25113 | F⁻, Δ(araD-araB)567, ΔlacZ4787(::rrnB-3), λ⁻, | Keio |
| rcsB⁻ | ΔrcsB770::kan, rph-1, Δ(rhaD-rhaB)568, hsdR514 | collection |
| BW25113 | F⁻, Δ(araD-araB)567, ΔlacZ4787(::rrnB-3), λ⁻, | Keio |
| rcsC⁻ | ΔrcsC771::kan, rph-1, Δ(rhaD-rhaB)568, hsdR514 | collection |
| W3110 | F⁻ λ⁻ rph-1 INV(rrnD, rrnE) | Prof. Pierre |
| | | Genevaux |
| W3110 dnaK⁻ | W3110 ΔDnaK52::CmR | Prof. Pierre |
| | | Genevaux |
| BW25113 rng⁻ | BW25113 Δrng-771::kan | Keio |
| | | collection |
| ENS134 | BL21(DE3), lacZ::Tn10, | Prof. A. |
| | malPpA534::PT71acZ::RBSIamB-Arg5 | Carpousis |
| ENS134-2 | ENS134 rne131 | Prof. A. |
| | | Carpousis |
| ENS134-10 | ENS134 rneΔ10 | Prof. A. |
| | | Carpousis |
| ENS134-14 | ENS134 rneΔ14 | Prof. A. |
| | | Carpousis |
| ENS134-17 | ENS134 rneΔ17 | Prof. A. |
| | | Carpousis |
| ENS134-18 | ENS134 rneΔ18 | Prof. A. |
| | | Carpousis |
| ENS 134-21 | ENS 134 rneΔ21 | Prof. A. |
| | | Carpousis |
| ENS134-22 | ENS 134 rneΔ22 | Prof. A. |
| | | Carpousis |
| ENS134-23 | ENS 134 rneΔ23 | Prof. A. |
| | | Carpousis |
| ENS134-24 | ENS 134 rneΔ24 | Prof. A. |
| | | Carpousis |
| C41(DE3) | F⁻ ompT gal dcm lon hsdSB(rB− mB−) λ(DE3 [lacI | Lucigen |
| | lacUV5-T7 gene 1 ind1 sam7 nin5]) with | |
| | modifications described by Kwon et al and | |
| | Schlegel et al. | |
| C43(DE3) | F⁻ ompT gal dcm lon hsdSB(rB− mB−) λ(DE3 [lacI | Lucigen |
| | lacUV5-T7 gene 1 ind1 sam7 nin5]) with | |
| | modifications described by Kwon et al. | |
| Lemo21(DE3) | F⁻ ompT gal dcm lon hsdSB(rB− mB−) λ(DE3 [lacI | New England |
| | lacUV5-T7 gene 1 ind1 sam7 nin5]) pLemo | Biolabs |
| SuptoxD | F⁻ λ⁻ Δ(ara-leu)7697 [araD139]B/r Δ(codB-lacI)3 | This work |
| | galK16 galE15 e14⁻ mcrA0 relA1 rpsL150(Str^{R}) | |
| | spoT1 mcrB1 hsdR2(r⁻m⁺) pSuptoxD | |
| SuptoxR | F⁻ λ⁻ Δ(ara-leu)7697 [araD139]B/r Δ(codB-lacI)3 | This work |
| | galK16 galE15 e14⁻ mcrA0 relA1 rpsL150(Str^{R}) | |
| | spoT1 mcrB1 hsdR2(r⁻m⁺) pSuptoxD | |

TABLE 3
Plasmids used in this work

| | | | Origin of | |
|---|---|---|---|---|
| Plasmid | Protein expressed | Marker | replication | Source |
| pBAD30BR2-KanR | FLAG-BR2-TEV-KanR | Amp^{R} | ACYC | This work |
| pBADBR2-GFP | FLAG-BR2-TEV-GFP-His₈ | Amp^{R} | ACYC | Skretas et al. |
| pASKBR2-GFP | FLAG-BR2-TEV-GFP-His₈ | Amp^{R} | ColE1 | Link et al. |
| pASK(KanR)BR2-GFP | FLAG-BR2-TEV-GFP-His₈ | Kan^{R} | ColE1 | This work |
| pASKBR2 | FLAG-BR2-His₈ | Amp^{R} | ColE1 | Link et al. |
| pASKCB1-GFP | FLAG-CB1-TEV-GFP-His₈ | Amp^{R} | ColE1 | Link et al. |
| pASKCB2-GFP | FLAG-CB2-TEV-GFP-His₈ | Amp^{R} | ColE1 | Link et al. |
| pASKNKR1-GFP | FLAG-NKR1-TEV-GFP-His₈ | Amp^{R} | ColE1 | Link et al. |
| pASKMotA-GFP | FLAG-MotA-GFP- His₈ | Amp^{R} | ColE1 | This work |
| pASKSapC-GFP | FLAG-SapC-GFP-His₈ | Amp^{R} | ColE1 | This work |
| pASKGarP-GFP | FLAG-GarP-GFP-His₈ | Amp^{R} | ColE1 | This work |
| pASKTcyL-GFP | FLAG-TcyL-GFP-His₈ | Amp^{R} | ColE1 | This work |
| pASKGsiC-GFP | FLAG-GsiC-GFP-His₈ | Amp^{R} | ColE1 | This work |
| pASKArtM-GFP | FLAG-ArtM-GFP- His₈ | Amp^{R} | ColE1 | This work |
| pASKMdfA-GFP | FLAG-MdfA-GFP- His₈ | Amp^{R} | ColE1 | This work |
| pASKCstA-GFP | FLAG-CstA-TEV-GFP- His₈ | Amp^{R} | ColE1 | Skretas et al. |
| pASKYidC-GFP | FLAG-YidC-TEV-GFP- His₈ | Amp^{R} | ColE1 | This work |
| pASKSCD-GFP | FLAG-SCD-GFP-His₈ | Amp^{R} | ColE1 | This work |
| pASKNTR1(D03)-GFP | FLAG-NTR1(D03)-GFP-His₈ | Amp^{R} | ColE1 | This work |
| pASKNTR1(D03)-TrxA | FLAG-NTR1(D03)-TrxA-His₆ | Amp^{R} | ColE1 | This work |
| pETBR2-GFP | FLAG-BR2-GFP-His₈ | Kan^{R} | ColE1 | This work |
| pETCB2-GFP | FLAG-CB2-GFP-His₈ | Kan^{R} | ColE1 | This work |
| pETMotA-GFP | FLAG-MotA-GFP-His₈ | Kan^{R} | ColE1 | This work |
| pASKBR2-EGFP | FLAG-BR2-TEV-EGFP-His₆ | Amp^{R} | ColE1 | This work |
| pASKNTR1(D03)-EGFP | FLAG-NTR1(D03)-TEV-EGFP-His₆ | Amp^{R} | ColE1 | This work |
| pASKSapC-EGFP | FLAG-SapC-TEV-EGFP-His₆ | Amp^{R} | ColE1 | This work |
| ASKA library | All known E. coli proteins | Cm^{R} | ColE1 | Kitagawa et al |
| pSuptoxR | RraA-His₈ | Cm^{R} | ACYC | This work |
| (pBAD33RraA) | | | | |
| pBAD33RraB | RraB-His₈ | Cm^{R} | ACYC | This work |
| pBAD30DjlA | DjlA-His₈ | Amp^{R} | ACYC | This work |
| pSuptoxD | DjlA-His₈ | Cm^{R} | ACYC | This work |
| (pBAD33DjlA) | | | | |
| pBAD33DnaJ | DnaJ-His₈ | Cm^{R} | ACYC | This work |
| pBAD33CbpA | CbpA-His₈ | Cm^{R} | ACYC | This work |
| pBAD33HscB | HscB-His₈ | Cm^{R} | ACYC | This work |
| pBAD33DjlB | DjlB-His₈ | Cm^{R} | ACYC | This work |
| pBAD33DjlC | DjlC-His₈ | Cm^{R} | ACYC | This work |
| pBAD33DjlA(M16R) | DjlAM16R-His₈ | Cm^{R} | ACYC | This work |
| pBAD33DjlA(H233Q) | DjlAH233Q-His₈ | Cm^{R} | ACYC | This work |
| pBAD33DjlAΔJ | DjlAΔJ-His₈ | Cm^{R} | ACYC | This work |
| pBAD33DjlAΔcentral | DjlAΔcentral-His₈ | Cm^{R} | ACYC | This work |
| pBAD33DjlAΔTMD | DjlAΔTM-His₈ | Cm^{R} | ACYC | This work |
| pBAD33DjlAJDnaJ | DjlAJDnaJ-His₈ | Cm^{R} | ACYC | This work |

Since the selected genes may be ameliorating cytotoxicity because they lower the levels of BR2 transcription and/or translation, we monitored BR2 accumulation in the absence and presence of the selected genes by measuring the fluorescence of MC1061 cells overexpressing a C-terminal BR2 fusion with green fluorescent protein (GFP) (FIG. 1A). It has been previously demonstrated that the fluorescence of E. coli cells expressing membrane protein-GFP fusions correlates well with the amount of membrane-integrated recombinant membrane protein and they have been utilized in high-throughput screens for identifying factors that enhance bacterial membrane protein production. Fifteen of the tested clones demonstrated BR2-GFP fluorescence which was at least as high as that demonstrated by wild-type cells overexpressing randomly selected ASKA genes, thus indicating that suppression of BR2-induced toxicity in these clones does not arise from inhibition of BR2 production. DNA sequencing of the ASKA plasmids for these fifteen clones revealed two genes as potential suppressors of MP toxicity: (i) djlA, the gene encoding for the membrane-bound DnaK co-chaperone DjlA, and (ii) rraA, the gene encoding for RraA, an inhibitor of the mRNA-degrading activity of the E. coli RNaseE. djlA was present in 4/15 sequenced clones and was isolated both from the 0 and 0.01 mM IPTG screens, while rraA in 11/15 sequenced clones and was isolated from the 0.1 mM IPTG screen.

Having established the toxicity-suppressing effects of DjlA and RraA on MP production, we refer to the utilized djlA- and rraA-encoding pBADB33 vectors as pSuptoxD (suppressor of toxicity—DjlA) and pSuptoxR (suppressor of toxicity—RraA), respectively (Table 3), and the E. coli strains co-expressing these effectors as SuptoxD and SuptoxR, respectively (Table 2).

### Example 2: SuptoxD and SuptoxR Accumulate Enhanced Levels of Membrane-Embedded BR2 with the Correct N^{out}-Cⁱⁿ Topology

It has been previously demonstrated that the fluorescence of E. coli cells expressing MP-GFP fusions correlates well with the amount of membrane-integrated recombinant MP and it has been utilized in high-throughput screens as a readout for identifying factors that enhance bacterial MP production. Based on this, we evaluated the levels of cellular BR2 accumulation in the generated strains by comparing the individual cell fluorescence of E. coli MC1061 (wild-type; WT), SuptoxD, and SuptoxR cells overexpressing BR2-GFP under the same conditions. Very interestingly, a large increase in individual cell fluorescence was recorded when BR2-GFP was produced in SuptoxD and SuptoxR (FIG. 1D). This suggests that the generated strains may also have the ability to accumulate increased cellular amounts of recombinant BR2, despite the fact that they were not directly selected for this property. Indeed, Western blot analysis of clarified lysates revealed that SuptoxD and SuptoxR cells can accumulate markedly increased amounts of BR2-GFP on a per cell basis compared to WT E. coli (FIG. 1E).

In order to investigate whether SuptoxD and SuptoxR can accumulate increased amounts of BR2 protein that is properly embedded in the bacterial membrane, we first utilized a FLAG-BR2 fusion (FIG. 1A, right). Suppression of BR2-induced toxicity, as well as the greatly enhanced per-cell accumulation of membrane-integrated receptor in SuptoxD and SuptoxR, was evident also for this construct, despite the absence of the C-terminal GFP tag (FIGS. 1F and G). To examine whether BR2 is inserted in the bacterial inner membrane with the correct N^{out}-Cⁱⁿ topology, we tested the exposure of the N-terminal BR2 tail to the bacterial periplasmic space. Spheroplasts derived from the same cells were labeled with an Alexa Fluor 647-conjugated anti-FLAG antibody and their fluorescence was recorded. As shown in FIG. 1H, red fluorescence was significantly enhanced in SuptoxD and SuptoxR cells, indicating that more BR2 with the correct N^{out} topology accumulates in the generated strains compared to WT E. coli. Finally, we tested whether the produced BR2 protein acquires a correct Cⁱⁿ topology. For this, we turned back to the BR2-GFP fusion, since MP-GFP fusions have been used extensively to study the localization of the termini of MPs of unknown topology. As GFP is unable to fold properly in the bacterial periplasm where it remains non-fluorescent, the fluorescence of E. coli cells expressing MP-GFP fusions indicates (i) a C-terminal localization for the GFP-tagged terminus when fluorescence is recorded or (ii) an N-terminal localization for the GFP-tagged terminus when fluorescence is absent. Production of a C-terminal BR2-GFP fusion in SuptoxD and SuptoxR resulted in a large increase in cellular fluorescence compared to WT E. coli (FIG. 1D), thus confirming that the C-terminal tail of the receptor is also properly localized in the bacterial cytoplasm. Taken together, these results demonstrate that SuptoxD and SuptoxR produce markedly enhanced amounts of properly membrane-embedded BR2 on a per-cell basis. Combination of the two positive effects of BR2 production in SuptoxD and SuptoxR, namely suppression of BR2-induced toxicity and enhancement in cellular BR2 accumulation, resulted in a dramatic enhancement in volumetric BR2 accumulation compared to WT E. coli (FIG. 1I).

### Example 3: SuptoxD and SuptoxR Broadly Enhance Recombinant Production for a Variety of Homologous and Heterologous MPs

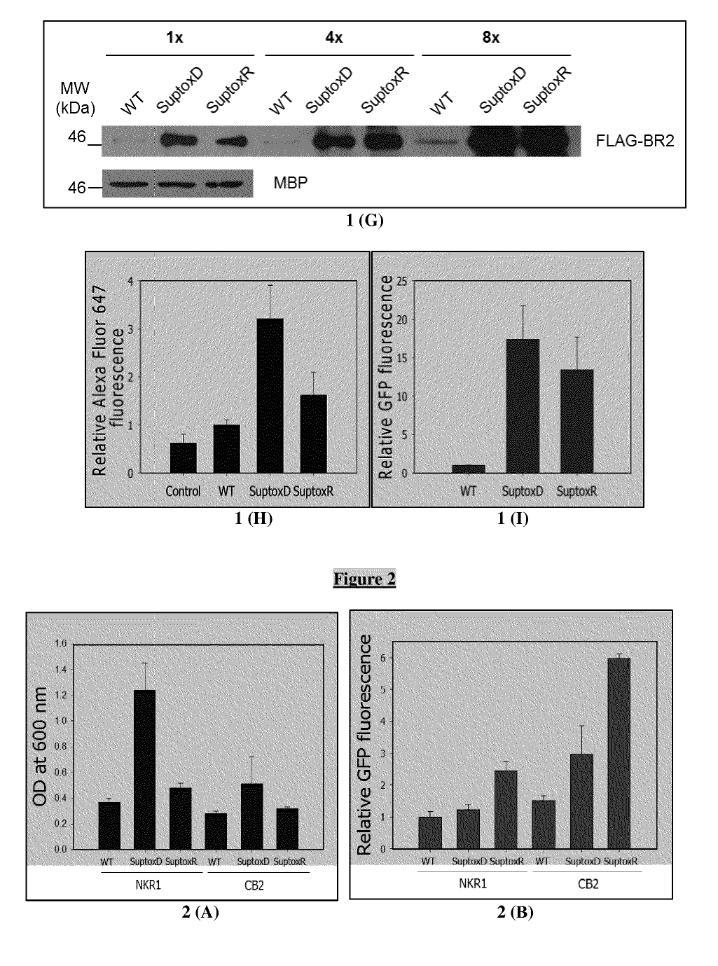

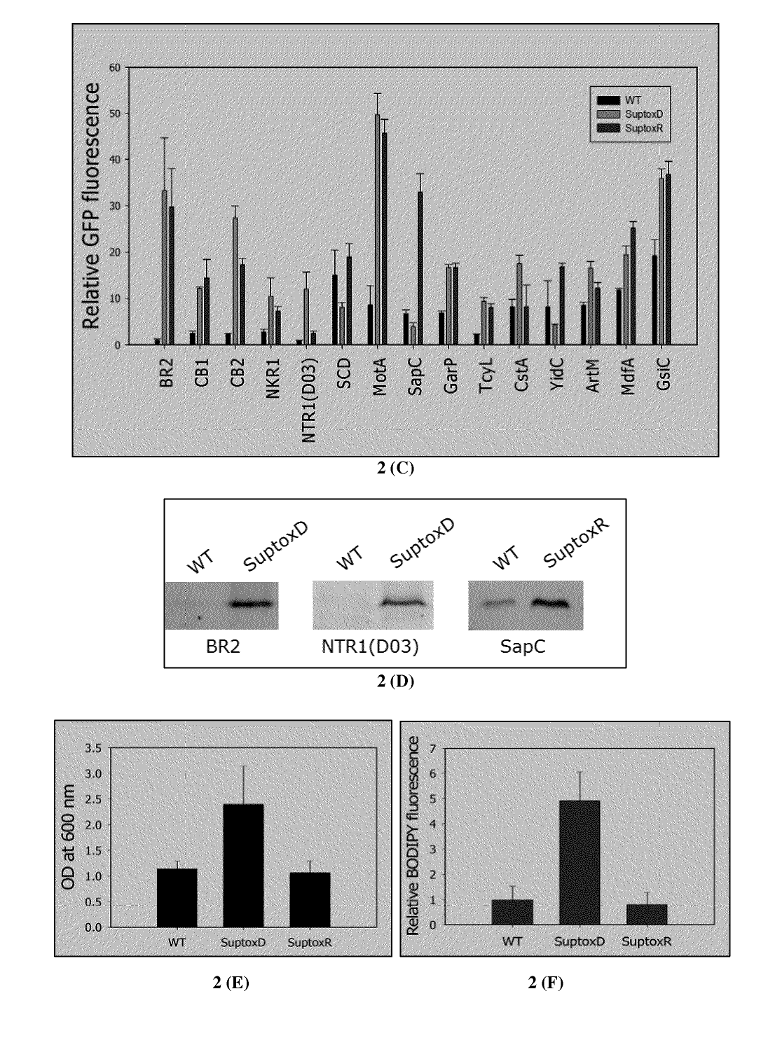

To examine whether the beneficial effects of SuptoxD and SuptoxR can be generally applied to other MPs as well, we first tested their effect on the toxicity and per-cell productivity of two additional human GPCRs, the neurokinin receptor 1 (NKR1) and the peripheral cannabinoid receptor (CB2) (Table 1). Overexpression of NKR1-GFP and CB2-GFP fusions in SuptoxD and SuptoxR resulted in marked increases in final culture densities, as well as levels of individual cell fluorescence for both receptors compared to WT E. coli (FIGS. 2A and B), thus demonstrating that the beneficial effects of these strains are not limited to the production of BR2. Combination of the toxicity-suppressing and cellular productivity-promoting effects on NKR1 and CB2 production resulted in a dramatic 2.6- to 11.5-fold enhancement in volumetric accumulation of both receptors in SuptoxD and SuptoxR (FIG. 2C).

To examine further the ability of SuptoxD and SuptoxR to enhance MP production broadly, we extended the panel of MPs to be tested to include additional integral MPs of bacterial or mammalian origin, characterized by different sizes, number of trans-membrane helices, topologies, and biochemical properties (Table 1). All proteins were expressed as C-terminal fusions with GFP and total MP accumulation was monitored by measuring bulk fluorescence of equal culture volumes. Remarkably, all tested MPs accumulated at greatly increased levels either in SuptoxD, SuptoxR, or both, compared to WT E. coli (FIG. 2C), thus demonstrating that our engineered strains can broadly act as general enhancers of recombinant MP production.

### Example 4: SuptoxD and SuptoxR Enhance the Production of Well-Folded Recombinant MPs

In order to investigate the folding quality of the overproduced MPs in the engineered strains, we analyzed by SDS-PAGE and in-gel fluorescence the production of three MPs, two eukaryotic (BR2, NTR1(D03)) and one prokaryotic (SapC), as C-terminal fusions with the enhanced green fluorescence protein (EGFP) in WT and either SuptoxD or SuptoxR cells, depending on which strain was found previously to be the most efficient producer for each MP. Previous studies have shown that in-gel fluorescence provides a quantitative measure of the amount of properly folded protein for bacterially overexpressed MP-EGFP fusions. This analysis revealed that all tested MP-EGFP fusions embedded in the membrane of SuptoxD and SuptoxR cells exhibit high levels of fluorescence (FIG. 2D), indicating that all tested MPs produced in our engineered strains adopt a well-folded conformation. Furthermore, in all cases the production of fluorescent, well-folded MP-GFP fusion was markedly enhanced in SuptoxD or SuptoxR compared to WT E. coli (FIG. 2D). Thus, SuptoxD and SuptoxR not only enhance the overall MP accumulation, but appear to assist the folding pathway of the recombinantly produced MP as well.

To assess whether these increases coincide with the production of more functional MP, we utilized NTR1(D03), a previously engineered variant of the rat neurotensin receptor 1. As shown in FIG. 2C, NTR1(D03)-GFP fluorescence indicated that NTR1(D03) accumulation is markedly enhanced in SuptoxD, while its production in SuptoxR has only a marginal impact (FIG. 2C). NTR1(D03) production as a fusion with thioredoxin 1 (TrxA) in SuptoxD resulted in a 2-fold enhancement in final biomass and a 5-fold increase in cellular labeling by a fluorescent conjugate of the neurotensin peptide 8-13 (NT(8-13)) with dipyrromethene boron difluoride (BODIPY) (FIGS. 2E and F). Analysis of the levels of accumulated membrane-embedded protein by Western blotting and densitometry revealed a 5.5-fold increase in the accumulation of membrane-embedded NTR1(D03)-TrxA in SuptoxD compared to WT cells (FIG. 2G), in full agreement with the fluorescent ligand binding experiments. Since the fluorescence of E. coli cells overexpressing NTR1 and labeled with BODIPY-NT(8-13) has been found to correlate well with the amount of functional overexpressed receptor, we estimate that NTR1(D03) production in SuptoxD results in an approximately 10-fold volumetric enhancement in accumulation of functional receptor.

### Example 5: Comparison of the MP-Producing Capabilitites of SuptoxD and SuptoxR with Commercial Strain Frequently Utilized for Recombinant MP Production

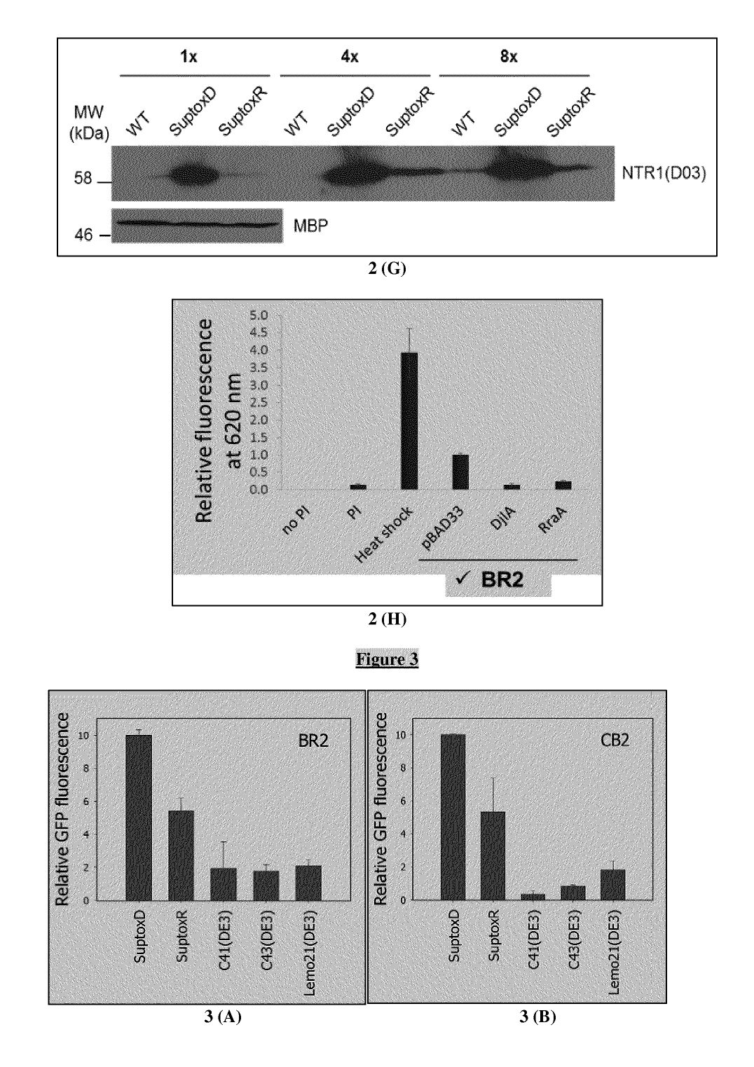

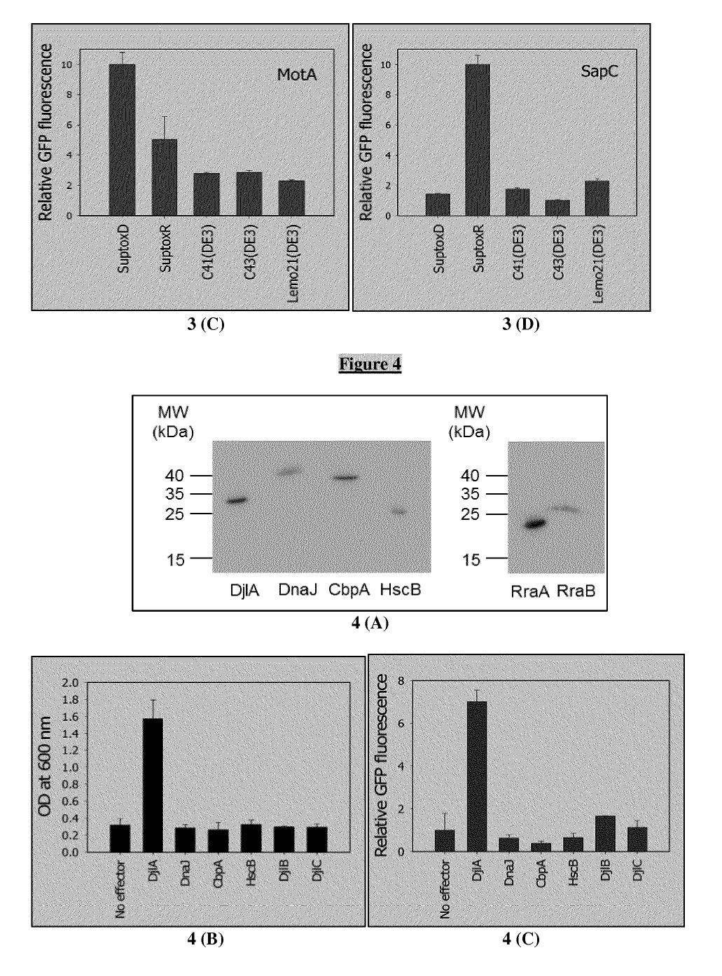

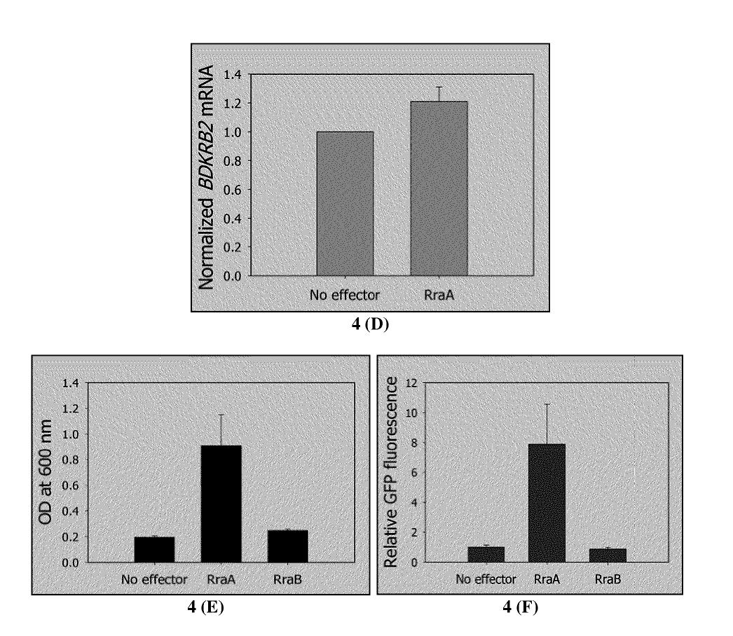

Finally, we went on to compare the MP-producing capabilities of SuptoxD and SuptoxR with the commercial E. coli strains C41(DE3), C43(DE3), and Lemo21(DE3) (Table 2), which are frequently utilized for the production of recombinant MPs and other toxic proteins. To make this comparison, two prokaryotic (MotA and SapC) and two eukaryotic (BR2 and CB2) MPs were cloned as C-terminal fusions with GFP into the T7 promoter-based vector pET-28a(+), and the resulting constructs were transformed into C41(DE3), C43(DE3), and Lemo21(DE3). For Lemo21(DE3), we first determined the optimal concentration of L-rhamnose that maximized volumetric production of each MP according to the manufacturer's instructions. Volumetric accumulation for each protein in the different strains was compared by measuring cell fluorescence of equal culture volumes. Either SuptoxD, or SuptoxR, or both, were found to accumulate greatly increased levels of recombinant protein for all tested MPs compared to the three frequently utilized commercial strains (FIG. 3).

### Example 6: DjlA is Unique in its Ability to Enhance Recombinant MP Production—DjlA and RraA Function Independently of One Another to Enhance Recombinant MP Production

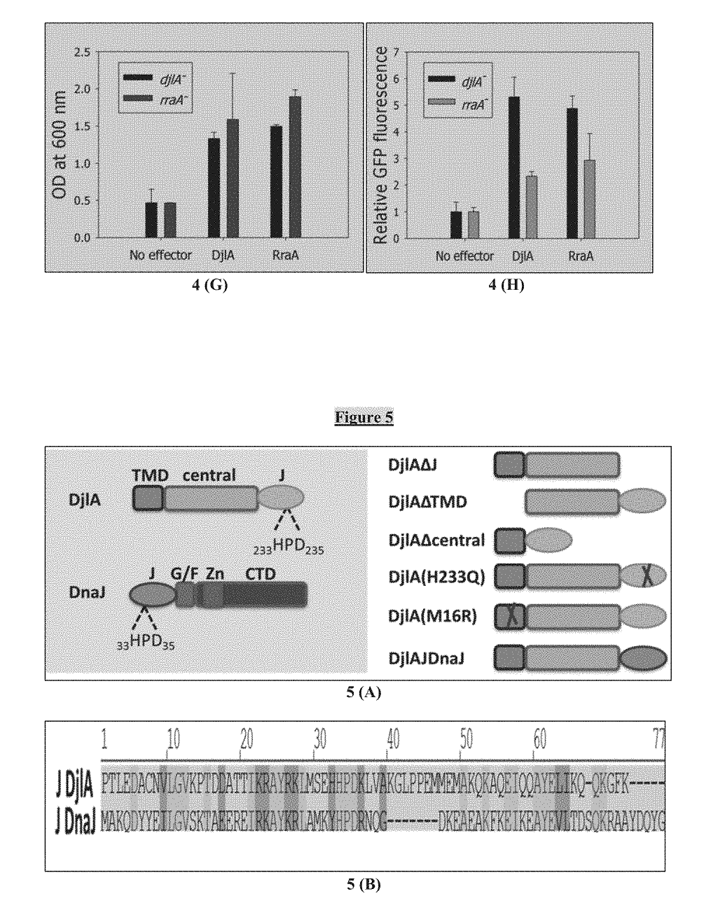

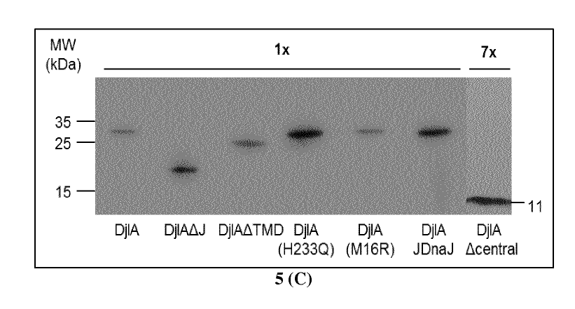

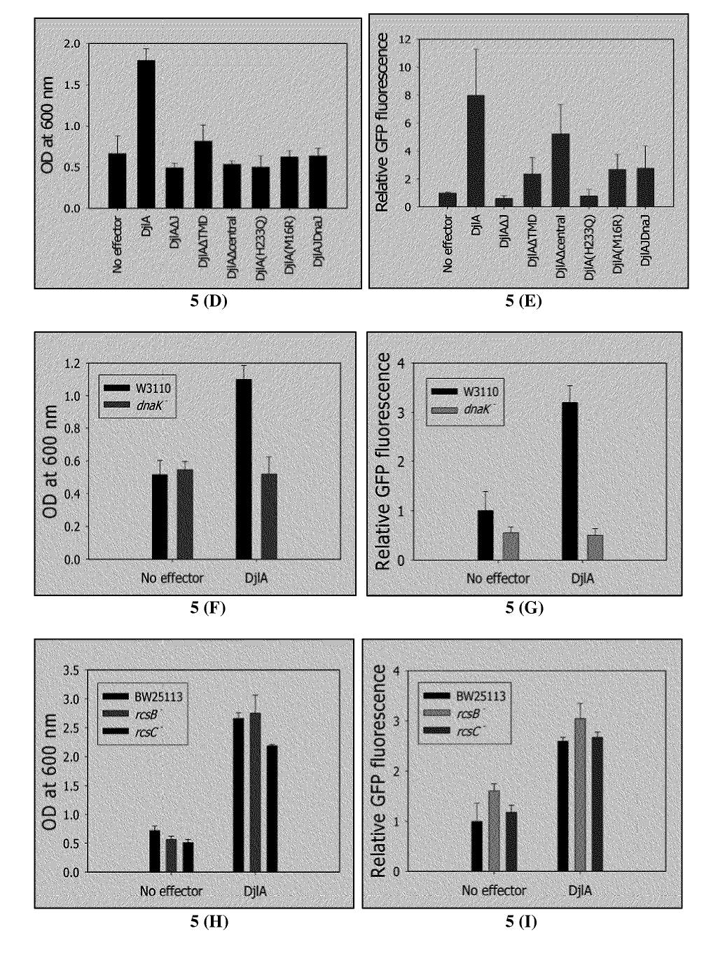

DjlA is a membrane-bound DnaK co-chaperone containing a C-terminal J domain, which is essential for interaction with DnaK. E. coli encodes for two additional DnaK co-chaperones, DnaJ and CbpA, which also interact with DnaK via their J domains, as well as for three additional J domain proteins (JDPs), HscB, DjlB, and DjlC, which are not known to interact with DnaK. In order to test whether other E. coli JDPs can exert similar effects, their genes were cloned along with a C-terminal 8×His tag into pBAD33 and their effect on BR2 accumulation was evaluated as above. Apart from DjlB and DjlC, whose production in membrane-bound form is known to be problematic in E. coli and could not be detected by Western blotting using an anti-polyHis antibody, all other JDPs accumulated at detectable levels (FIG. 4A, left). Very interestingly, no other JDP was found to act as a suppressor of BR2-induced toxicity or as an enhancer of BR2 production (FIGS. 4B and C). This suggests that DjlA is unique among its analogues in its ability to facilitate bacterial recombinant MP production and provides an explanation about why no other JDPs were identified in our initial screen.

Next, to test whether DjlA and RraA act independently, we monitored the effect of djlA or rraA co-expression on BR2-GFP cytotoxicity and production on a rraA⁻ or djlA⁻ genetic background, respectively. The positive effects of RraA and DjlA on BR2 production and final cell density were observed regardless of the presence of functional DjlA and RraA, respectively, thus demonstrating that the effect of each protein on MP production occurs through a mechanism that does not involve the other (FIGS. 4G and H).

### Example 7: Full-Length and Membrane-Bound DjlA is Required for Exerting the Observed Beneficial Effects on MP Production

DjlA is composed of three functional domains: (i) An N-terminal trans-membrane domain (TMD), which has been shown to participate in dimerization and possibly facilitates DjlA-mediated signaling as well; (ii) a central domain of unknown function (central); and (iii) a C-terminal J domain (J) that mediates the interaction with DnaK (FIG. 5A). To assess the role of these different domains in the observed effects, we generated truncation variants of DjlA lacking each functional domain individually: the TMD (DjlAΔTMD variant), the central (DjlAΔcentral), or the C-terminal J domain (DjlAΔJ) (FIG. 5A). Furthermore, we generated point mutants and domain-swapped variants in functionally critical areas of the protein: (i) the M16R variant, which carries a substitution located in the TMD that leads to reduced activation of the regulation of capsular synthesis (Rcs) response, a well-established effect of djlA overexpression, while retaining its capability for dimerization and proper insertion into the E. coli cytoplasmic membrane; (ii) the H233Q substitution, whose equivalent mutations in the highly conserved HPD motif (FIG. 5A) of the J domain abrogate its functional interaction with DnaK; and (iii) the DjlAJDnaJ variant, where the J domain of DjlA was replaced with that of DnaJ (FIG. 5A). These domains share 31% identity and 44% similarity (FIG. 5B) and have partially overlapping functions. C-terminally His-tagged versions of these variants were tested for their ability to alleviate BDKRB2 overexpression toxicity and/or increase BR2 accumulation. All variants were tested in a djlA⁻ background in order to avoid possible dimerization with WT DjlA. Despite the fact that the generated variants were found to accumulate at similar levels (with the exception of DjlAΔcentral, which accumulated at lower but detectable levels) (FIG. 5C), no variant was capable of suppressing BR2-induced toxicity (FIG. 5D). Very interestingly, all variants containing a functional J domain could enhance cellular BR2 accumulation, albeit to a significantly smaller extent compared to WT DjlA (FIG. 5E). Taken together, these results make a number of points. First, they show that full-length and membrane-bound WT DjlA is required for suppressing MP-induced toxicity. Second, as demonstrated by the effects of the DjlA(H233Q) variant, a functional interaction with DnaK appears to be required for both DjlA-mediated suppression of MP-induced toxicity and cellular enhancement of MP production. Indeed, co-expression of djlA in a dnaK⁻ background resulted in complete loss of its ability to suppress MP-induced toxicity and promote MP production (FIGS. 5F and G). Third, DjlA-promoted cellular enhancement of MP production is primarily mediated by its J domain; still, a functional J domain is not sufficient for the observed effect, as indicated by the inability of all other E. coli JDPs to enhance BR2 production (FIGS. 4B and C). Fourth, as suggested by the impact of the M16R substitution, the beneficial effects of djlA co-expression on MP production could be occurring due to activation of the Rcs response through the RcsB/RcsC two-component system. DjlA, however, was efficient in suppressing MP-induced cytotoxicity and enhancing MP accumulation irrespective of the presence of functional RcsB or RcsC (FIGS. 5H and I), thus demonstrating that its effects are not mediated due to activation of the Rcs response.

### Example 8: The Observed Beneficial Effects of RraA on Recombinant MP Production are Mediated by the Action of RNase E and Occur Due to Inhibition of its Ribonucleolytic Activity Upon rraA Overexpression

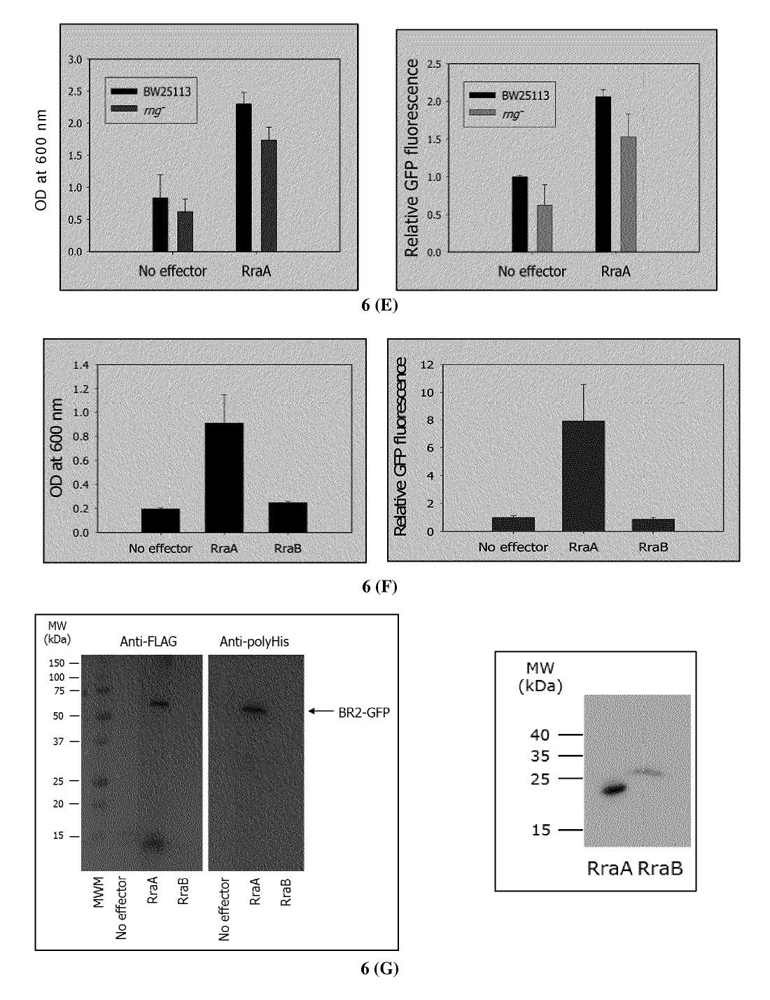

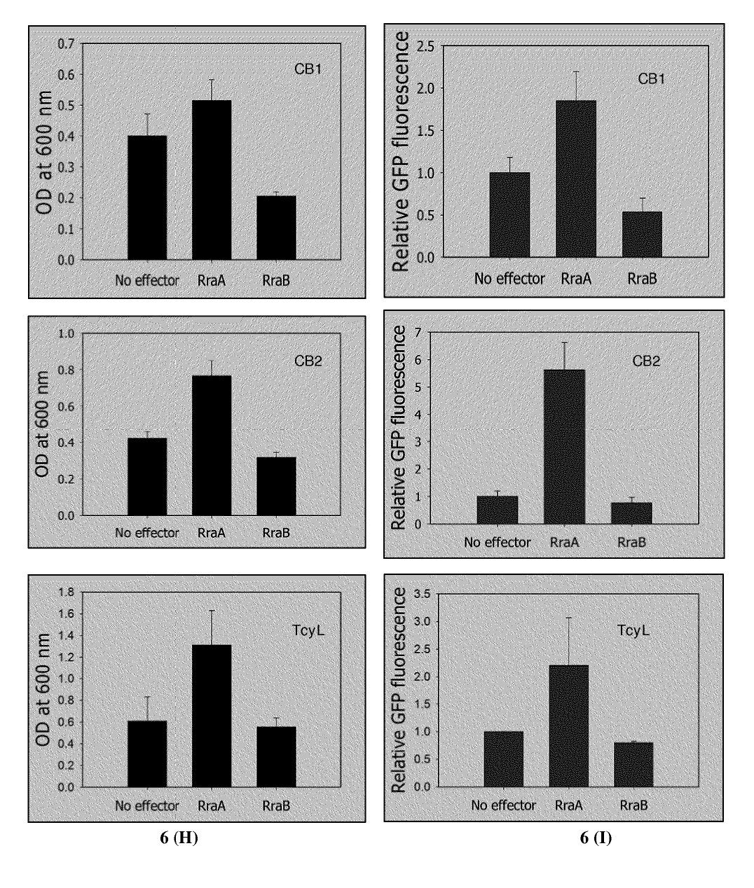

Our second identified enhancer of recombinant MP production, RraA (Regulator of ribonuclease activity A), is known to act as a regulator of the mRNA-degrading activity of RNase E, and rraA overexpression has been found to affect the levels of more than 2,000 different mRNAs in E. coli. RNase E, in turn, is a large, 1061-amino-acid-long endonuclease, which constitutes the major enzyme of mRNA turnover in E. coli, and is essential for viability. In this organism and other bacteria, RNase E-mediated mRNA degradation can be carried out by this endonuclease alone or by a multi-enzyme complex called the RNA degradosome, in which RNase E forms the core and is complemented by three additional enzymes: polynucleotide phosphorylase (PNPase), the DEAD-box helicase RhlB, and the glycolytic enzyme enolase. RNase E is composed of two main functional domains: (i) an N-terminal tetrameric catalytic domain, which is responsible for its ribonucleolytic activity, and (ii) an intrinsically disordered C-terminal domain (CTD), which contains the main regions that mediate interactions with the other components of the degradosome and other regulatory proteins, such as RraA (FIG. 6a).

To study the potential involvement of RNase E in the observed beneficial effects of rraA overexpression on recombinant MP production, we tested the performance of a series of previously generated E. coli strains carrying different chromosomal me mutations, which encode for RNase E truncation variants with deletions in various parts of its CTD (FIG. 6a), on BR2-induced toxicity, cellular BR2 accumulation and total volumetric BR2 accumulation. The ribonucleolytic activity of RNase E is essential for E. coli viability and, thus, me null strains or strains lacking the N-terminal catalytic domain of RNase E cannot be generated. The utilized strains have been found to exhibit variable levels of cellular RNase E-mediated ribonucleolytic activity, which in all cases but one (stain ENS134-10) are reduced—at least for the particular transcripts that have been tested—compared to cells producing full-length RNase E. As a result of this analysis, we found that the RraA-mediated enhancement of MP production was diminished, or could no longer be detected at all, in the majority of the strains encoding the different me alleles (FIGS. 6b-d). More specifically, statistically significant RraA-mediated enhancements in the levels of cellular and volumetric BR2 accumulation could not be observed in the vast majority of strains with depleted RNase E activity encoding mutant me alleles, which contained smaller (e.g. strains ENS134-22 and ENS134-24), larger (e.g. strains ENS134-14, ENS134-18 and ENS134-23), or complete deletions of the CTD of RNase E (strain ENS134-2) (FIGS. 6b-d). Furthermore, we observed that in a number of the tested strains with depleted RNase E activity, levels of final biomass after BDKRB2 overexpression, or BR2 accumulation, or both, were significantly enhanced compared to the parental strain with wild-type RNase E activity (compare, for example, BR2-GFP fluorescence for equal culture volumes in the strains ENS134-2, 21 and 22 [no effector] with that in ENS134 [no effector]; P=0.003, 0.0002 and 0.0009, respectively) (FIGS. 6b-d). These observations indicate that the observed beneficial effects of RraA on recombinant MP production are mediated by the action of RNase E and occur due to inhibition of its ribonucleolytic activity upon rraA overexpression.

Since the deletion of small or larger parts of the CTD or of this entire domain resulted in the disappearance of the beneficial effects of RraA on recombinant MP production, our results suggest that its action is mediated primarily via the CTD of RNase E (FIGS. 6a-d). This observation is in agreement with the fact that RraA has been found to interact with the RNase E CTD and to regulate the ribonucleolytic activity of RNase E primarily via its association with this domain (FIG. 6a). Very interestingly, however, the presence of the entire RNase E CTD does not appear to be absolutely necessary for RraA-mediated enhancement of recombinant MP production, since deletion of a large portion of the C-terminal region of the CTD (strain ENS134-10) that includes regions of importance for interactions with other components of the E. coli RNA degradosome, such as PNPase, —but not with RraA—did not affect significantly the MP production-promoting ability of RraA (FIGS. 6a-d). Again, this observation is in agreement with the fact that the main sites of interaction of RraA with RNase E are in the areas of the CTD lying at and between the AR1 and AR2 (FIG. 6a), both of which are located upstream of the site of interaction with PNPase.

In contrast, the ribonucleolytic activity of the RNase E paralog RNase G, does not appear to be involved in the MP production-promoting effects of RraA, as rraA co-expression in an E. coli strain carrying a complete deletion of the RNase G-encoding gene rng resulted in both suppression of MP-induced toxicity and enhancement of cellular MP production at levels, which were similar to those observed in wild-type (WT) E. coli (FIG. 6e). In support of this observation, elimination of RNase G activity did not reduce the levels of BR2-induced toxicity or enhance cellular BR2 accumulation (FIG. 6e; compare the rng and parental strains in the absence of effector co-expression), contrary to what was observed previously for a number of the me mutants (FIGS. 6b-d). RNase G is, in terms of domain architecture, a minimal version of RNase E, comprising only a ribonuclease domain and lacking completely a separate functional domain resembling the RNase E CTD (FIG. 6a, bottom) but possesses partially overlapping functions with RNase E, as rng overexpression has been found to be effective in the complementation of an me null E. coli mutant. However, and consistently with the fact that RNase G does not possess a regulatory domain but only a catalytic domain, the ribonucleolytic activity of RNase G is not known to be regulated by other protein factors, such as RraA, a feature which is also in line with the herein observed effects. Taken together, our results demonstrate that the impact of rraA overexpression on recombinant MP production is mediated by the action of RNase E and not by other paralogous E. coli ribonucleases, such as RNase G.

Apart from RraA, the E. coli genome encodes for a second similar regulator of RNase E activity, termed RraB, which modulates the RNase E-mediated decay of a partially overlapping, but mostly distinct set of transcripts compared to RraA. Consistently with this, rraB co-expression was found ineffective in suppressing BR2-induced toxicity and in enhancing BR2 accumulation (FIGS. 6f and g), despite the fact that RraB accumulates at detectable levels (FIG. 6g, right) and globally inhibits the RNase E-mediated decay of hundreds of transcripts when overexpressed in E. coli. The inability of RraB to promote recombinant MP production was not only observed in the case of BR2, but also when additional recombinant MP targets were considered: CB1, CB2, and the membrane subunit of the E. coli cystine/cysteine ABC transporter TcyL (Table 1; FIGS. 6h and i). Thus, like DjlA, RraA is also unique among similar proteins in its ability to promote recombinant MP production in E. coli. Again, this observation provides an explanation about why no other bacterial modulators of RNase E activity were identified in our initial screen for suppressors of MP-induced toxicity.

The fact that rraB overexpression does not suppress BR2-induced toxicity or enhance BR2 accumulation indicates that a reduction of RNase E activity in a generic fashion may not be sufficient to mimic all of the beneficial effects of rraA co-expression on recombinant MP production. Indeed, the majority of the previously tested me mutant strains exhibiting lower levels of RNase E activity compared to E. coli with WT RNase E activity, were found to be markedly less efficient in simultaneously suppressing the toxicity caused by BDKRB2 overexpression and promoting BR2 production levels compared to WT E. coli overexpressing rraA. For example, strains ENS134-14, ENS134-17, ENS134-18, ENS134-21, ENS134-23 and ENS134-24 have been found to exhibit reduced levels of RNase E activity, yet this depleted RNase E activity results in about 3-fold lower volumetric MP accumulation to E. coli encoding WT RNase E and overexpressing rraA (FIG. 6d; ENS134[+RraA] vs ENS134-14, 17, 18, 21, 23 and 24[no effector]: P=0.009, 0.006, 0.004, 0.01, 0.006, and 0.007, respectively). On the contrary, in two of the tested me mutant strains, ENS134-2 and ENS134-22, also known to exhibit decreased ribonucleolytic activity through RNase E, volumetric MP production was found to occur at levels as high or higher than WT E. coli ENS134 overexpressing rraA (FIG. 6d). Taken together, our results indicate that RNase E activity needs to be inhibited in a highly specialized manner so that both MP overexpression-induced toxicity can be suppressed and cellular MP accumulation can be enhanced. This observation is not entirely surprising considering that the transcriptomes of E. coli cells under conditions of RNase E depletion due to mutations in me or due to rraA or rraB overexpression have been found to be, to a smaller or a larger extent, different.

### Example 9: Membrane Protein Overexpression in Liquid Cultures

E. coli cells freshly transformed with the appropriate expression vector(s) were used for all protein production experiments. Single bacterial colonies were used to inoculate liquid LB cultures containing the appropriate combination of antibiotics (100 μg/mL ampicillin, 40 μg/mL chloramphenicol or 50 μg/mL kanamycin (Sigma)). These cultures were used with a 1:50 dilution to inoculate fresh LB cultures with 0.01 (MC1061 and SuptoxD cells) or 0.2% L(+)-arabinose (SuptoxR cells), which were grown at 30° C. to an optical density at 600 nm (OD₆₀₀) of ˜0.3-0.5 with shaking. The temperature was then decreased to 25° C. and after a temperature equilibration period of 10-20 min, membrane protein expression was induced by the addition of 0.2 μg/ml anhydrotetracycline (aTc) (Sigma) overnight. As described also in previous studies, in certain cases MP-producing cultures exhibited abnormally high levels of growth after overnight induction of MP overexpression, a phenomenon which occurs due to chromosomal and/or plasmid mutations that inhibit the otherwise toxic process of MP production. This phenomenon was more frequently observed when MP overexpression occurred in the absence of our effectors. In the cases where non-expressing bacterial cells managed to outcompete the expressing cells and abnormal growth was recorded, the samples were discarded.

### Example 10: Membrane Isolation

Total membrane fractions were isolated from 500 ml LB cultures (2 L for in-gel fluorescence experiments). Cells were harvested by centrifugation and re-suspended in 10 ml of cold lysis buffer (20 ml for in-gel fluorescence experiments) (300 mM NaCl, 50 mM NaH₂PO₄, 15% glycerol, 5 mM dithiothreitol, pH 7.5). The cells were lysed by brief sonication steps on ice and the resulting lysates were clarified by centrifugation at 10,000×g for 15 min. The supernatant was then subjected to ultracentrifugation on a Beckman 70Ti rotor at 42,000 rpm (130,000×g) for 1 h at 4° C. The resulting pellet was finally resuspended in 10 ml of cold lysis buffer and homogenized.

### Example 11: Western Blot and in-Gel Fluorescence Analysis

Proteins samples were analyzed by sodium dodecyl sulfate-polyacrylamide gel electrophoresis (SDS-PAGE) on 10 or 15% gels. In-gel fluorescence was analyzed on a UVP ChemiDoc-It2 Imaging System equipped with a CCD camera and a GFP filter, after exposure for about 3 sec. For Western blotting, proteins were transferred to polyvinylidene fluoride (PVDF) membranes (Merck) for 50 min at 12 V on a semi-dry blotter. Membranes were blocked with 5% non-fat dry milk in Tris-buffered saline containing 0.1% Tween-20 (TBST) for 1 h at room temperature. After washing with TBST three times, membranes were incubated with the appropriate antibody dilution in TBST containing 0.5% non-fat dried milk at room temperature for 1 h. The utilized antibodies were a mouse monoclonal anti-polyhistidine antibody (Sigma) at 1:2,500 dilution, a mouse monoclonal anti-FLAG antibody (Sigma) at 1:1,000 dilution, a mouse anti-GFP antibody at 1:20,000 dilution (Clontech) and a mouse anti-MBP antibody (New England Biolabs) at 1:2,500 dilution, all conjugated with horseradish peroxidase. After washing with TBST three times, the proteins were visualized on X-ray film with SuperSignal West Pico chemiluminescent substrate (Pierce).

### Example 12: Spheroplast Generation and Labelling

Cells corresponding to 3 ml of a culture with OD₆₀₀ equal to 1 were re-suspended in 350 μl of an aqueous solution containing 0.75 M sucrose, 0.1 M Tris, pH 8. The samples were mixed by vortexing while 700 μl of 1 mM EDTA, pH 8 were being added dropwise, and the resulting cell suspension was incubated for 4 min at room temperature. 35 μl of 20 mg/ml lysozyme in PBS were then added and the samples were mixed by slow rotation on a rotating wheel for 20 min. After the addition of 50 μl MgCl₂ 0.5 M, the samples were kept on ice for 10 min. Spheroplasts were pelleted at 10,000×g for 10 min at 4° C. and resuspended in 0.5 mL PBS. 167 μl of sample were spun down at 10,000×g for 10 min at 4° C., the supernatant was discarded and the pellet was resuspended in a 200 μl PBS solution containing a 1:400 dilution of an Alexa Fluor 647-conjugated anti-FLAG antibody (Cell Signaling Technology) and rotated on a rotating wheel for 3 h. The spheroplasts were then washed with 500 μl PBS, spun down at 10,000×g for 10 min at 4° C. and resuspended in 100 μl PBS. The cell suspension was then transferred to a black 96-well plate and bulk fluorescence was measured using a TECAN SAFIRE2 plate reader.

### Example 13: Bulk Fluorescence Measurements

Cells corresponding to 0.25 OD₆₀₀ units were harvested and re-suspended in 100 μl PBS. The cell suspension was then transferred to a black 96-well plate and after fluorophore excitation at 488 nm for GFP or 647 nm for Alexa Fluor 647, fluorescence was measured at 510 nm for GFP and 670 nm for Alexa Fluor 647 using a TECAN SAFIRE2 plate reader.

1. A method of producing a recombinant polypeptide in a host cell, wherein the host is genetically modified so as to express elevated levels of DjlA and/or RraA, or variants thereof, relative to the expression of said protein in a wild-type strain.

2. A method of producing a recombinant polypeptide in a host cell as claimed in claim 1 comprising the steps of: (a) providing a nucleic acid comprising a sequence for the recombinant polypeptide and a nucleic acid sequence for either djlA and/or rraA, or variants thereof, operably linked to a promoter into an expression system; and (b) expressing the nucleic acid sequences of step (a) under conditions that allow expression of the polypeptide, thereby producing a recombinant polypeptide and either DjlA and/or RraA and, optionally, purifying the recombinant polypeptide.

3. A method as claimed in claim 1 wherein the recombinant polypeptide in the absence of elevated levels DjlA and/or RraA is toxic to the host.

4. A method as claimed in claim 1 wherein the recombinant polypeptide in the absence of elevated levels DjlA and/or RraA is produced at unsatisfactory levels by the host.

5. A method as claimed in claim 3 where in the recombinant polypeptide is a membrane protein.

6. A method as claimed in claim 1 wherein the host, naturally occurring or genetically modified, is selected from the prokaryotic hosts Escherichia coli, Lactococcus lactis, Bacillus subtilis, Pseudomonas aeruginosa, Erwinia carotovora, Salmonella choleraesuis, Agrobacterium tumefaciens, Chromobacterium violaceum, Salmonella or the eukaryotic hosts Saccharomyces cerevisae, Pichia pastoris, Schizosaccharomyces pombe, Kluyveromyces lactis, CHO, NS0, HEK293, HeLa, Sf9, tobacco, rice, and Leishmania tarentolae.

7. A method as claimed in claim 1 wherein the variant is selected from one of the following:
(i) functional variants of DjlA and RraA that work as well or even better, or not significantly any worse, than DjlA and RraA themselves;
(ii) silent changes to the nucleotide sequence of djlA and rraA that do not change the amino acid sequence expressed; and
(iii) homologous amino acid sequences of DjlA and/or RraA from other organisms.

8. A method as claimed in claim 7, wherein the variant is a homologous amino acid sequence of DjlA and/or RraA from other organisms and DjlA is selected, for example, from homologues found in a broad spectrum of Gram-negative bacteria, such as Legionella species, Shigella flexneri, Shewanella putrefaciens, Salmonella typhimurium, Vibrio cholerae, Coxiella burnetii, Haemophilus influenza, Yersinia pestis and Yersinia enterocolitica, and RraA is selected, for example, from close homologs found in various bacteria, archaea, proteobacteria, and plants, such as Mycobacterium tuberculosis, Vibrio vulnificus, Thermus thermophilus, Vibrio cholera, and Arabidopsis thaliana.

9. A method as claimed in claim 1 of transforming a host cell with (a) a nucleic acid comprising a sequence for a recombinant polypeptide and a sequence for either djlA, or a variant thereof, and/or rraA, or a variant thereof, operably linked to a promoter followed by expressing the nucleic acid of step (a), thereby producing the recombinant polypeptide and either DjlA and/or RraA and in the transformed cell.

10. A vector as claimed in claim 1 for transforming a host cell comprising a nucleic acid sequence for a recombinant polypeptide and a nucleic acid sequence for either djlA and/or rraA, or a variant thereof, operably linked to a promoter.

11. A transformed host cell as claimed in claim 1 comprising a nucleic acid sequence encoding the recombinant polypeptide and either DjlA and/or RraA, or a variant thereof.

12. A host cell as claimed in claim 1, wherein the host is genetically modified so as to express elevated levels of DjlA and/or RraA, or variants thereof, relative to the expression of said protein in a wild-type strain.

13. A host cell as claimed in claim 1, wherein the host is genetically modified so as to express improved variants of DjlA and/or RraA, relative to those expressed by the wild-type strain.

14. A host cell as claimed in claim 11, wherein the host is genetically modified so as to express variants of the ribonuclease RNase E with depleted ribonucleolytic activity.


SEQUENCE LISTING (6 sequences)

SEQ ID NO: 1 - DNA, 816 bp, Escherichia coli
atgcagtattggggaaaaatcattggcgtggccgtggccttactgatgggcggcggcttttggggcgtagtgttaggcctgttaattggccatatgtttgataaagcccgtagccgtaaaatggcgtggttcgccaaccagcgtgagcgtcaggcgctgttttttgccaccacttttgaagtgatggggcatttaaccaaatccaaaggtcgcgtcacggaggctgatattcatatcgccagccagttgatggaccgaatgaatcttcatggcgcttcccgtactgcggcgcaaaatgcgttccgggtgggaaaatcagacaattacccgctgcgcgaaaagatgcgccagtttcgcagtgtctgctttggtcgttttgacttaattcgtatgtttctggagatccagattcaggcggcgtttgctgatggttcactgcacccgaatgaacgggcggtgctgtatgtcattgcagaagaattagggatctcccgcgctcagtttgaccagtttttgcgcatgatgcagggcggtgcacagtttggcggcggttatcagcagcaaactggcggtggtaactggcagcaagcgcagcgtggcccaacgctggaagatgcctgtaatgtgctgggcgtgaagccgacggatgatgcgaccaccatcaaacgtgcctaccgtaagctgatgagtgaacaccatcccgataagctggtggcgaaaggtttgccgcctgagatgatggagatggcgaagcagaaagcgcaggaaattcagcaggcatatgagctgataaagcagcagaaagggtttaaatga

SEQ ID NO: 2 - protein, 271 aa, Escherichia coli
MQYWGKIIGVAVALLMGGGFWGVVLGLLIGHMFDKARSRKMAWFANQRERQALFFATTFEVMGHLTKSKGRVTEADIHIASQLMDRMNLHGASRTAAQNAFRVGKSDNYPLREKMRQFRSVCFGRFDLIRMFLEIQIQAAFADGSLHPNERAVLYVIAEELGISRAQFDQFLRMMQGGAQFGGGYQQQTGGGNWQQAQRGPTLEDACNVLGVKPTDDATTIKRAYRKLMSEHHPDKLVAKGLPPEMMEMAKQKAQEIQQAYELIKQQKGFK

SEQ ID NO: 3 - DNA, 486 bp, Escherichia coli
atgaaatacgatacttccgagctttgtgacatctatcaagaagatgttaacgtcgtggaaccgctgttctccaactttggcggacgggcgtcgtttggcggacaaataatcacggtaaaatgtttcgaggacaacgggttgctgtacgatctgctcgaacagaatggccgtggtcgtgttcttgtcgtcgatggcggtggttctgttcgtcgcgcactggtcgatgctgaactggcgcgtctggcagtacaaaatgaatgggaaggtctggtcatttacggcgcggtgcgtcaggtagatgacctggaagagttggatatcggcatccaggcgatggcggcaattccggttggtgccgctggcgaaggcattggcgaaagcgatgtccgcgtcaattttggcggtgtcaccttcttctccggcgaccatctttatgccgacaataccgggattattctttcagaagatccgctggatattgaatga

SEQ ID NO: 4 - protein, 161 aa, Escherichia coli
MKYDTSELCDIYQEDVNVVEPLFSNFGGRASFGGQIITVKCFEDNGLLYDLLEQNGRGRVLVVDGGGSVRRALVDAELARLAVQNEWEGLVIYGAVRQVDDLEELDIGIQAMAAIPVGAAGEGIGESDVRVNFGGVTFFSGDHLYADNTGIILSEDPLDIE

SEQ ID NO: 5 - protein, 71 aa, Escherichia coli
PTLEDACNVLGVKPTDDATTIKRAYRKLMSEHHPDKLVAKGLPPEMMEMAKQKAQEIQQAYELIKQQKGFK

SEQ ID NO: 6 - protein, 70 aa, Escherichia coli
MAKQDYYEILGVSKTAEEREIRKAYKRLAMKYHPDRNQGDKEAEAKFKEIKEAYEVLTDSQKRAAYDQYG